SEQUENCE LISTING (2 sequences)

SEQ ID NO: 1 - protein, 390 aa, Canis sp.
MLSRKGIIPEEYVLTRLAEDPAEPRYRARERRARFVSKNGNCNVAHKNIREQGRFLQDVFTTLVDLKWPHTLLIFTMSFLCSWLLFAMVWWLIAFAHGDLAPGEGTAVPCVTSIHSFSSAFLFSIEVQVTIGFGGRMVTEECPLAILILIVQNIVGLMINAIMLGCIFMKTAQAHRRAETLIFSKHAVIAVRHGRLCFMLRVGDLRKSMIISATIHMQVVRKTTSPEGEVVPLHQVDIPMENGVGGNSIFLVAPLIIYHVIDANSPLYDLAPSDLHHHQDLEIIVILEGVVETTGITTQARTSYLADEILWGQRFVPIVAEEDGRYSVDYSKFGNTIKVPTPLCTARQLDEDRSLLDALTLASARGPLRKRSVAVAKAKPKFSISPDSLS

SEQ ID NO: 2 - RNA, 1173 nt, Canis sp.
  source 1..1173: mol_type = transcribed RNA
atgctgtcccgaaaaggcatcatccccgaggagtacgtgctcacgcggctggccgaggaccccgcagagccccggtaccgagctcgggagcggagggcccgcttcgtgtccaagaacggcaattgcaacgtagcgcacaagaacatccgggagcagggccgattcctgcaggacgtattcaccacgctggtggacctcaagtggccacacacactgctcatctttaccatgtccttcctgtgcagctggctgctctttgccatggtctggtggctcatcgccttcgcccacggtgacctggcccccggtgagggcactgcggtgccctgtgtcaccagcatccactctttttcatcygccttccttttctccattgaggtccaggtgaccattggctttggcgggcgcatggtgaccgaggagtgcccgctggccatcttgatcctcattgtgcagaacatcgtggggctcatgatcaatgccatyatgctgggctgcatcttcatgaagacggcccaggcccatcggcgggccgagaccctcatcttcagcaagcatgcggtcatcgcagtgcgccacggccgcctctgcttcatgctgcgcgtcggtgacctccgcaagagcatgatcatcagcgccaccatccacatgcaggtggtgcgcaagaccaccagccccgagggtgaagtggtgcccctacaccaggtggacatccccatggagaacggtgtgggcggcaacagcatcttcctggtagctcctctcatcatctaccacgtcattgatgccaacagtccactctatgacctggcacccagcracctgcatcaccatcaagacctggagatcattgtcatcctggagggcgtggtggaaaccacgggcatcactacccaggcccgcacctcctacctggccgatgagatcctytggggccagcgctttgtacccatcgtggccgaggaggatggccgctactctgtggactactccaaatttggcaacaccatcaaagtgcccacgccactctgcacggcccgccagcttgatgaggaccgcagcctgctggacgccctgaccctcgcctcggcccgcggtcccctgcgcaagcgcagtgtggctgtggccaaggccaaacccaagtttagcatctctccggattccctgtcctga

(19) United States
(12) Patent Application Publication
(10) Pub. No.: US 2026/0199438 A1
(43) Pub. Date: Jul. 16, 2026

(54) METHOD AND TREATMENT FOR DIABETES IN DOGS

(71) Applicant: THE ROYAL VETERINARY COLLEGE, London (GB)

(72) Inventors: Lucy DAVISON, London (GB); Marsha WALLACE, London (GB); Brian CATCHPOLE, London (GB); Frances ASHCROFT, Oxford (GB); Natascia VEDOVATO, Oxford (GB)

(21) Appl. No.: 19/103,720

(22) PCT Filed: Aug. 4, 2023
(86) PCT No.: PCT/GB2023/052067

§ 371 Date: Feb. 13, 2025

(30) Foreign Application Priority Data
Aug. 16, 2022 (GB) 2211936.6

(51) Int. Cl. A61K 38/28; A61K 31/64; A61P 3/10; C12Q 1/6883

(52) U.S. Cl. CPC A61K 38/28; A61K 31/64; A61P 3/10; C12Q 1/6883; C12Q 2600/106; C12Q 2600/156

(57) ABSTRACT

The disclosure relates to a method of screening a dog for a predisposition to diabetes mellitus, and a method of determining the potential of a dog to produce progeny that are genetically predisposed to diabetes mellitus. The disclosure also relates to a method of selecting a treatment for diabetes mellitus in a dog, a method of preventing, delaying or treating diabetes mellitus in a dog using an oral hypoglycaemic drug, and a composition comprising an oral hypoglycaemic drug for use in a method of preventing, delaying or treating diabetes mellitus in a dog. The disclosure further concerns a method of preventing or delaying diabetes mellitus in a dog using an anti-hyperglycaemic diet or by means of neutering, and an anti-hyperglycaemic diet for use in a method of preventing or delaying diabetes mellitus in a dog.

### FIELD OF THE DISCLOSURE

The disclosure relates to a method of screening a dog for a predisposition to diabetes mellitus, and a method of determining the potential of a dog to produce progeny that are genetically predisposed to diabetes mellitus. The disclosure also relates to a method of selecting a treatment for diabetes mellitus in a dog, a method of preventing, delaying or treating diabetes mellitus in a dog using an oral hypoglycaemic drug, and a composition comprising an oral hypoglycaemic drug for use in a method of preventing, delaying or treating diabetes mellitus in a dog. The disclosure further concerns a method of preventing or delaying diabetes mellitus in a dog using an anti-hyperglycaemic diet or by means of neutering, and an anti-hyperglycaemic diet for use in a method of preventing or delaying diabetes mellitus in a dog.

### BACKGROUND

Diabetes mellitus is characterised by chronically elevated blood glucose as a consequence of insufficient insulin secretion or sensitivity. As well as diabetes being common in humans, veterinarians recognise spontaneous diabetes mellitus in the canine species with an approximate prevalence of 1 in 300 dogs. The median age of canine diabetes onset is 7 years, but in some breeds, such as the Labrador retriever, onset may be as early as 8 weeks of age. Affected dogs are typically dependent on twice-daily insulin injections, which can be burdensome for owners and unpleasant for the animal.

The insulin-dependence and pancreatic beta cell loss typically observed in canine diabetes suggests some similarity to human autoimmune type 1 diabetes mellitus (TID) rather than type 2 diabetes mellitus (T2D), which is characterised by insulin resistance and beta cell dysfunction. However, unlike human TID, evidence for an autoimmune pathogenesis in canine diabetes is very limited.

In humans, rare monogenic forms of diabetes have been identified which present in neonatal and early adult life. Approximately 50% of mutations of these mutations are found in genes encoding the pore-forming (KCNJ11, Kir6.2) and regulatory (ABCC8, SUR1) subunits of the ATP-sensitive potassium (K_{ATP}) channel. A common variant in KCNJ11 also predisposes to type 2 diabetes in humans. The K_{ATP} channel plays a key role in glucose-stimulated insulin secretion from pancreatic beta-cells by regulating membrane electrical excitability. Glucose uptake and metabolism by beta-cells generates ATP, which closes K_{ATP} channels and thereby triggers membrane depolarisation, electrical activity, calcium influx and insulin secretion. Activating K_{ATP} channel mutations impair the ability of ATP to close the channel, which inhibits insulin secretion.

While the genetic and pathophysiological basis of human diabetes is well characterised, the basis of canine disease remains uncertain. A better understanding of canine diabetes may provide for improved treatments that are more acceptable to the owner and animal, and breeding programmes that mitigate the likelihood of diabetic offspring.

### SUMMARY OF THE DISCLOSURE

The present inventors have identified the first diabetes mellitus-associated missense mutation in the canine KCNJ11 gene. The present inventors have further characterised this mutation as a monogenic cause of diabetes mellitus in dogs.

The mutation may be used to screen a dog for predisposition to diabetes mellitus. This may advantageously allow early diagnosis and treatment upon the development of clinical disease. Preventative measures may also be implemented before the development of clinical disease, e.g. in the pre-diabetic state. For instance, steps may be taken to minimise the occurrence of hyperglycemia in the dog. Hyperglycemia is toxic to insulin-producing pancreatic beta cells, and so minimizing the occurrence of hyperglycemia may help to preserve beta cells and/or their ability to produce insulin.

Human neonatal diabetes caused by KCNJ11 mutations is treated by administering oral hypoglycaemic drugs, such as sulphonylureas. Diabetic dogs possessing the KCNJ11 gene mutation disclosed herein may be treated in the same way. Such oral treatment is advantageous relative to traditional twice-daily injectable insulin therapy. For instance, oral drugs are simpler to administer and may be better tolerated by the dog. In addition, treatment with oral sulphonylureas may reduce blood glucose fluctuations, lower HbA1c, and facilitate meal-stimulated insulin secretion (by enabling incretin action). The present disclosure therefore provides a beneficial new treatment regimen for canine diabetes. The disclosure also provides a way to identify dogs likely to benefit from the regimen, by means of genetic testing for the KCNJ11 gene mutation.

The KCNJ11 gene mutation disclosed herein may also be used to determine the potential of a dog to produce progeny that are genetically predisposed to diabetes mellitus. This enables the identification carrier dogs, who may be excluded from the breeding pool to reduce the incidence of offspring having diabetes.

Accordingly, the disclosure provides a method of screening a dog for a predisposition to diabetes mellitus, comprising determining whether the genotype of the dog's KCNJ11 gene comprises a D274N allele. The disclosure further provides:
a method of determining the potential of a dog to produce progeny that are genetically predisposed to diabetes mellitus, comprising determining whether the genotype of the dog's KCNJ11 gene comprises a D274N allele;
a method of selecting a treatment for diabetes mellitus in a dog, comprising determining whether the genotype of the dog's KCNJ11 gene comprises a D274N allele, and selecting a treatment based on the determined genotype;
a method of preventing, delaying or treating diabetes mellitus in a dog whose KCNJ11 gene comprises one or more D274N alleles, comprising administering an oral hypoglycaemic drug to the dog;
a composition comprising an oral hypoglycaemic drug for use in a method of preventing, delaying or treating diabetes mellitus in a dog whose KCNJ11 gene comprises one or more D274N alleles, wherein the method comprises administering the oral hypoglycaemic drug to the dog;
a method of preventing or delaying diabetes mellitus in a dog whose KCNJ11 gene comprises one or more D274N alleles, comprising: (a) providing the dog with an anti-hyperglycaemic diet; and/or (b) neutering the dog, wherein the dog is female and neutering is performed before the dog's first oestrus cycle; and
an anti-hyperglycaemic diet for use in a method of preventing or delaying diabetes mellitus in a dog whose KCNJ11 gene comprises one or more D274N alleles.

### BRIEF DESCRIPTION OF THE FIGURES

FIG. 1: UK Labrador retrievers and KCNJ11 rs851344999 association with diabetes. (A) Choropleth map of diabetic Labrador retriever samples submitted to the study by referring veterinary practices in the United Kingdom, with representation from 33 of 41 UK regions (left). Doughnut plot showing samples in the study by phenotype and sex; M=Male, F=Female, NA=Not Available (right). (B) Stacked bar plot showing association of KCNJ11 rs851344999 genotype with diabetes mellitus in Labrador retrievers; χ² p=0.004838.

FIG. 2: Functional studies of the D274N mutation. (A) Representative recordings of whole-cell currents measured in oocytes expressing wild-type (WT) or homomeric D274N mutant (D274N) K_{ATP} channels in control solution, following addition of 3 mM Na-azide and after subsequent addition of 0.5 mM tolbutamide (Tolb). (B) WT and D274N current amplitudes recorded in control solution (Ctrl: •, ∘), 3 mM Na-azide (Az: ▴, Δ) and 3 mM Na-azide+0.5 mM tolbutamide (Tb: ▪, □). (C) The whole-cell current in control solution minus the tolbutamide-blocked current for WT (•, n=10) and D274N (∘, n=17) channels, ****P<0.0001 (Student t-test). (B,C) Mean±SEM plus individual data points. (D) Mean tolbutamide block of WT (light green bar, n=10) and D274N (dark green bar, n=17) KAIP channels, calculated as the percentage block of the azide-induced current (ns: not significant, Student t-test). The mean tolbutamide block for 19 human PNDM mutations is shown for comparison (white bar). The horizontal grey bar indicates the level of tolbutamide block that in human patients carrying activating KCNJ11 mutations separates those who respond to sulphonylurea treatment from those wo do not.

FIG. 3: D274N mutation reduces ATP inhibition of the K_{ATP} channel. (A,B) Representative recordings of wild-type (A, WT) or homomeric D274N mutant (B, D274N) KAIP currents from inside-out patches excised from transfected HEK cells exposed to different [ATP]. Holding potential-60 mV. The dashed line indicates the zero-current level. (C,D) Dose-response relationships for ATP inhibition of WT (▪, •) or D274N (□, ∘) K_{ATP} channels, in the absence (C) or presence (D) of Mg²⁺. The KAIP current in the presence of nucleotide (I) is expressed as a fraction of that in its absence (I_{C}, control). The lines are the best fit of the Hill equation to the mean data (C: WT, IC₅₀=10.2 μmol/l, h=1.2, n=7; D274N, IC₅₀=15.4 μmol/l, h=1.3, n=5. D: WT, IC₅₀=18.6 μmol/l, h=1.1, n=8; D274N, IC₅₀=29.0 μmol/l, h=1.0, n=7). (E,F) IC₅₀s for current inhibition derived from Hill fits to individual dose-response relationships. Mean±SEM plus individual data points (E, WT, IC₅₀=10: ±1 μmol/l, h=1.24±0.05, n=7; D274N, IC₅₀=15±1 μmol/l, h=1.19±0.06, n=5; F, WT, IC₅₀=19±2 μmol/l, h=1.12±0.04, n=8; D274N, IC₅₀=32±2 μmol/l, h=0.99±0.03, n=7), **P<0.01 (Student t-test). (G) Fraction of unblocked current at 3 mmol/l MgATP for WT (•) and D274N (∘) K_{ATP} channels, ***P<0.001 (Student t-test).

FIG. 4: Predicted effect of the D274N mutation on the Kir6.2 protein and its interaction with SUR1. (A) Overall tridimensional structure of the ATP sensitive potassium (KATP) channel, composed of four pore-forming subunits (Kir6.2—indigo, green, purple and yellow) and four regulatory sulfonylurea receptor SUR ATP-binding cassette subunits (SUR1—grey, teal, red and pink). The position of D274 is indicated by the black box. (B) Position of the ATP binding site in relation to D274. (C) and (D) Kir6.2 and SUR1 interface in the wildtype D274 and the mutated N274 protein. N274 does not impact the hydrogen bonds (black) and the cation-Pi interactions (red) between Kir6.2 H276 and H278 (green) and SUR1 R1352, S1356 and S1357 (pink). (E) Close-up view of D274 forming one hydrogen bond with A271 and two with Q279. (F) Substitution with the mutant N274 residue is predicted to disrupt one of the two polar contacts with Q279 and the polar contact with A271.

FIG. 5: UK Labrador retrievers and KCNJ11 rs851344999 association with diabetes. (A) Choropleth map with corresponding bar plot of Labrador retrievers diagnosed with diabetes mellitus as adults by average age across the United Kingdom. (B) Correlation matrix of Age, Weight, Insulin Dose, Fructosamine, and HbA1c in Labrador retrievers with adult onset of diabetes (top). Scatter plots of statistically significant correlations (bottom); Weight with Insulin Dose (R=0.45, p=9e-06); Fructosamine with HbA1c (R=0.59, p=3.7e-10). (C) Stacked bar plots showing a significant difference in the proportion of Neutered Female KCNJ11 rs851344999 heterozygotes (CT) with diabetes compared to Neutered Male heterozygotes (Fisher's p=0.0293) (top), while the proportion of neutered female Labrador retrievers with diabetes who are homozygous for the reference allele of KCNJ11 rs851344999 (CC) is not significantly different than homozygous neutered males (Fisher's p=0.811) (bottom).

FIG. 6: Histogram of percentage Labrador (2 copy dogs; whole study population). The histogram shows the percentage of Labrador retriever in dogs identified by the Wisdom Panel as being homozygous for rs851344999.

FIG. 7: Histogram of percentage Labrador (1 copy dogs; whole study population). The histogram shows the percentage of Labrador retriever in dogs identified by the Wisdom Panel as being heterozygous for rs851344999.

FIG. 8: Histogram of percentage Labrador (1 copy dogs; subset). The histogram shows the percentage of Labrador retriever in a subset of dogs identified by the Wisdom Panel as being heterozygous for rs851344999, and included in the Banfield Optimal Wellness Plans.

FIG. 9: Histogram of percentage Labrador (2 copy dogs; subset). The histogram shows the percentage of Labrador retriever in a subset of dogs identified by the Wisdom Panel as being homozygous for rs851344999, and included in the Banfield Optimal Wellness Plans.

FIG. 10: Ancestry information for 2 copy dogs. The figure provides full ancestry information for 10 representative dogs identified by the Wisdom Panel as being homozygous for rs851344999, and included in the Banfield Optimal Wellness Plans. A) Plot showing selected population. B) Table setting out ancestry. In the table, (B) indicates both sides of the family tree.

### DETAILED DESCRIPTION

It is to be understood that different applications of the disclosed methods and products may be tailored to the specific needs in the art. It is also to be understood that the terminology used herein is for the purpose of describing particular embodiments of the disclosure only, and is not intended to be limiting.

All publications, patents and patent applications cited herein, whether supra or infra, are hereby incorporated by reference in their entirety.

### General Definitions

Unless defined otherwise, technical and scientific terms used herein have the same meaning as commonly understood by a person skilled in the art to which this disclosure belongs.

As used in this specification and the appended claims, the singular forms “a”, “an”, and “the” include plural referents unless the content clearly dictates otherwise. Thus, for example, reference to “a cell” includes “cells”, reference to “an antisense oligonucleotide” includes two or more such antisense oligonucleotides, and the like.

In general, the term “comprising” is intended to mean including but not limited to. For example, the phrase “a composition comprising an oral hypoglycaemic drug” should be interpreted to mean that the composition contains an oral hypoglycaemic drug, but that the composition may contain additional nucleic acids.

In some aspects of the disclosure, the word “comprising” is replaced with the phrase “consisting of”. The term “consisting of” is intended to be limiting. For example, the phrase “a composition consisting of an oral hypoglycaemic drug” should be understood to mean that the composition contains an oral hypoglycaemic drug and no additional components.

### Screening for a Predisposition to Diabetes Mellitus

Disclosed herein is method of screening a dog for a predisposition to diabetes mellitus, comprising determining whether the genotype of the dog's KCNJ11 gene comprises a D274N allele.

As explained above, it may be advantageous to determine whether or not a dog is predisposed to diabetes mellitus. For example, knowledge that a dog is predisposed to diabetes mellitus may facilitate monitoring for the development of clinical disease. That is, the dog's owner and/or veterinarian can watch out for the onset of diabetes mellitus. This may allow early diagnosis and treatment, thereby improving the welfare of the dog. Early diagnosis and treatment may also improve clinical outcome, as sustained hyperglycemia negatively affect beta cell function and/or number. For instance, sustained hyperglycemia may impair insulin secretion, decrease insulin gene expression, and ultimately cause beta cell apoptosis. The loss of beta cells, or of beta cell function, may render the dog more dependent on insulin therapy, and/or cause more unstable disease.

Knowledge that a dog is predisposed to diabetes mellitus may also allow preventative measures to be implemented before the development of clinical disease. For instance, therapy may be initiated in the pre-diabetic state in order to minimise pathophysiological changes leading to clinical diabetes mellitus. Such therapy may, for example, aim to delay the onset of hyperglycemia, minimise the frequency of hyperglycaemic episodes, and/or reduce the magnitude of hyperglycemia. As set out above, hyperglycemia has adverse effects on beta cell number and function. Such adverse effects may contribute to the development of clinical diabetes mellitus. Therapy that counteracts hyperglycemia may therefore prevent or delay the onset of clinical diabetes mellitus. Such therapy may, for example, comprise administration of oral hypoglycaemic drugs (such as sulphonylureas) and/or dietary management.

### Dog

The method screens a dog for a predisposition to diabetes mellitus. The dog may be any canine individual, of any breed. In one aspect, the dog is a Labrador retriever or has Labrador retriever ancestry.

A dog with Labrador retriever ancestry may be defined as a dog that is descended from a Labrador retriever. A dog with Labrador retriever ancestry may possess one or more alleles inherited from a Labrador retriever. The one or more alleles may comprise an allele that is typically restricted to Labrador retrievers. The one or more alleles may comprise a D274N allele.

Dogs with Labrador retriever ancestry may, for example, include other retriever breeds, such as flat coat retrievers or golden retrievers. A dog with Labrador retriever ancestry may, for example, be cross-breed of a Labrador retriever and one or more other breeds of dog. Labrador cross-breeds may include labradoodles (Labrador x poodle), springadors (Labrador x springer spaniel), goldadors (Labrador x golden retriever), boradors (Labrador x border collie) and so on.

The dog may be of any age. The dog may, for example, be an adult. The dog may, for example be a juvenile or puppy.

The dog may be of any sex. For instance, the dog may be an entire (i.e. non-neutered) female. The dog may be an entire (i.e. non-neutered) male. The dog may be a neutered female. The dog may be a neutered male.

### Predisposition

The purpose of the method is to identify a predisposition to diabetes mellitus. A predisposition to diabetes mellitus may be defined as an increased risk of developing diabetes mellitus. A dog predisposed to diabetes mellitus may have an increased risk of developing diabetes mellitus relative to the population average. That is, a dog predisposed to diabetes mellitus may be more likely to develop diabetes mellitus than other dogs. For instance, a Labrador retriever predisposed to diabetes mellitus may be more likely to develop diabetes mellitus than other Labrador retrievers.

The predisposition that the method seeks to identify is a predisposition associated with D274N mutation of the canine KCNJ11 gene. Therefore, the predisposition is a genetic predisposition. Thus, the method screens the dog for a genetic susceptibility to diabetes mellitus. Other genetic predispositions to diabetes mellitus may exist, associated with other mutations and/or other genes.

### Diabetes Mellitus

Diabetes mellitus is a well-known disease in which blood glucose is chronically elevated as a consequence of insufficient insulin secretion or sensitivity. Clinical signs of diabetes mellitus are associated with hyperglycemia and, in dogs, may typically include increased drinking (polydipsia), increased urination (polyuria), and/or weight loss. Appetite may be increased or decreased. Canine diabetes mellitus may also be associated with the development of cataracts (i.e. clouding over of the lens of the eye), and/or recurrent infections such as urinary tract infections.

Canine diabetes mellitus may be diagnosed using the “ALIVE” criteria. Using the ALIVE criteria, diabetes mellitus may be diagnosed:
1) In a patient with a random (fasted or unfasted) blood glucose of greater than or equal to 200 mg/dl (11.1 mmol/l) with classic clinical signs of hyperglycaemia (with no other plausible cause) or hyperglycaemic crisis. In some cases, clinical signs may not have been reported by the owner. In cases with uncertainty over presence/absence of clinical signs diagnosis may be confirmed by repeat blood glucose measurement and/or documentation of alternative glycaemic parameters such as increased glycated proteins and/or glucosuria.
2) In some patients with fasting blood glucose of between 7 mmol/l and 11 mmol/l with or without clinical signs of hyperglcaemia or hyperglycaemic crisis. Diabetes mellitus is differentiated from stress hyperglycaemia by documentation of persistent fasting hyperglycaemia for more than 24 hours or increased glycated proteins.

Canine diabetes mellitus is typically type 1-like. That is, diabetes mellitus in the dog tends to be phenotypically similar to type 1 diabetes mellitus (TID) in humans. In humans, TID is characterised by insulin dependence, pancreatic beta cell dysfunction and/or pancreatic beta cell loss. Thus, the method of the disclosure may, for example, screen for a predisposition to diabetes mellitus that is characterised by (i) insulin dependence, (ii) pancreatic beta cell dysfunction, and/or (iii) pancreatic beta cell loss. The method of the disclosure may, for example, screen for a predisposition to diabetes mellitus that is characterised by (i); (ii); (iii); (i) and (ii); (i) and (iii); (ii) and (iii); or (i), (ii) and (iii)

### Determining Genotype

In order to screen for a predisposition to diabetes mellitus, the method comprises determining whether the genotype of the dog's KCNJ11 gene comprises a D274N allele.

The canine KCNJ11 gene is a known gene, having the NCBI gene ID 485401. The canine KCNJ11 gene encodes a protein, Kir6.2. The amino acid sequence of canine Kir6.2 is represented by SEQ ID NO: 1.

A D274N allele of canine KCNJ11 encodes a mutant Kir6.2 protein in which aspartate (D) at position 274 is substituted for asparagine (N). The position of this substitution is shown by underlining in SEQ ID NO: 1. A D274N allele may arise from a single nucleotide polymorphism that results in a thymine (T) in place of a cytosine (C) found in the canine reference genome Canis Familiaris 3.1. In other words, a D274N allele may arise from a C>T polymorphism in the canine KCNJ11 gene. The C>T polymorphism may be the polymorphism known as rs851344999. SEQ ID NO: 2 shows a primary transcript of KCNJ11 (KCNJ11-201) that comprises rs851344999 (see position 820, underlined). Nucleotide “N” at position 820 of SEQ ID NO: 2 may be G (the complement of the C nucleotide found at the corresponding position in the canine reference genome Canis Familiaris 3.1) or A (the complement of the T nucleotide found at the corresponding position in a D274N allele of canine KCNJ11). For completeness, SEQ ID NO: 2 contains a number of further “N” nucleotides at positions 357, 486 and 930 which may each be C or T, although these polymorphisms are not the subject of the present disclosure.

To determine whether the genotype of the dog's KCNJ11 gene comprises a D274N allele, the genotype of the KCNJ11 gene may be determined. Methods for determining genotype are well-known in the art. Any known genotyping method may be used to determining whether the genotype of the dog's KCNJ11 gene comprises a D274N allele.

Typically, the genotype of the KCNJ11 gene is determined by analysing a sample obtained from the dog. Analysis may occur in vitro. The sample may be any sample that contains genetic material from the dog. The sample may, for example, comprise cells from the dog. In one aspect, the sample is a blood sample. In another aspect, the sample is a tissue sample. Preferably, the tissue sample is one that may be obtained non-invasively. The tissue sample may, for example, comprise hair, skin cells, or mucosal cells. For instance, the tissue sample may be a buccal swab.

The genotype of the KCNJ11 gene may, for example, be determined by genome sequencing. In this case, the sequence of all or part of the dog's genome is determined. The determined sequence can then be examined to establish whether or not the KCNJ11 gene comprises a D274N allele. Numerous sequencing methods are known in the art, and may be used in the method of the disclosure. The sequencing may, for example, be Sanger sequencing. The sequencing may, for example, be next generation sequencing (NGS; e.g. Illumina). Next generation sequencing may also be known as high throughput sequencing. The sequencing may, for example, be long read sequencing. Examples of long red sequencing technologies include nanopore sequencing and single molecule real-time sequencing (SMRT; e.g. PacBio). The sequencing may, for example, be DNA nanoball sequencing (e.g. DNBSEQ).

The genotype of the KCNJ11 gene may, for example, be determined by single nucleotide polymorphism (SNP) genotyping. In the context of the present disclosure, SNP genotyping refers to methods that discriminate alleles that differ by a single base substitution, without the need to sequence the allele. SNP genotyping methods are known in the art and include 5′-nuclease allelic discrimination assays (e.g. TaqMan assays), restriction fragment length polymorphism analysis, allele-specific PCR (also known as KASP), and SNP arrays (a type of DNA microarray).

Genotyping of the dog's KCNJ11 gene may determine that: (1) the dog's KCNJ11 gene comprises two D274N alleles (i.e. that the gene/dog is homozygous for the D274N allele); (2) the dog's KCNJ11 gene comprises one D274N allele and one reference allele (i.e. that the gene/dog is heterozygous for the D274N allele); or (3) the dog's KCNJ11 gene comprises two reference alleles (i.e. that the gene/dog is homozygous for the reference allele). A reference allele is a KCNJ11 allele that encodes wild-type canine Kir6.2 that does not comprise a D274N mutation. The reference allele may encode canine Kir6.2 of SEQ ID NO: 1. The reference allele does not comprise a C>T polymorphism in the canine KCNJ11 gene that encodes a D274N mutation in canine Kir6.2.

### Link Between Genotype and Predisposition

The genotype of the dog's KCNJ11 gene may be used to determine a predisposition to diabetes mellitus. As demonstrated in the Example, there is a significant association between rs851344999 genotype and canine diabetes mellitus. Thus, dogs whose KCNJ11 gene comprises one or more D274N allele may be predisposed to diabetes mellitus.

In more detail, the Example demonstrates a monogenic recessive impact of the D274N mutation of KCNJ11 on diabetes mellitus phenotype. Accordingly, a KCNJ11 genotype homozygous for the D274N allele may indicate that the dog is predisposed to diabetes mellitus. In other words, a dog whose KCNJ11 comprises two D274N alleles may be predisposed to diabetes mellitus. Therefore, the method may comprise determining that the genotype of the dog's KCNJ11 gene comprises two D274N alleles, and thereby concluding that the dog is predisposed to diabetes mellitus.

Homozygosity for the D274N allele may be sufficient to confer a predisposition to diabetes mellitus. That is, diabetes mellitus may arise in dogs homozygous for the D274N allele in the absence of other diabetogenic factors. Homozygosity for the D274N allele may, for example, be causative of diabetes mellitus. The rationale for causation is explained in detail in the Example. In brief, KCNJ11 encodes the protein Kir6.2. As set out above, Kir6.2 is the pore-forming subunit of the ATP-sensitive potassium (K_{ATP}) channel. In pancreatic beta cells, glucose uptake generates ATP, which closes K_{ATP} channels. This ATP-mediated inhibition of K_{ATP} channels triggers membrane depolarisation, electrical activity, calcium influx and insulin secretion. The Example demonstrates that D274N-mutant K_{ATP} channels are less sensitive to ATP-mediated inhibition. Insulin secretion is therefore impaired in beta cells from dogs homozygous for the D274N allele of KCNJ11.

A KCNJ11 genotype heterozygous for the D274N allele may indicate that the dog is predisposed to diabetes mellitus. In other words, a dog whose KCNJ11 gene comprises one D274N allele and one reference allele may be predisposed to diabetes mellitus. Therefore, the method may comprise determining that the genotype of the dog's KCNJ11 gene comprises one D274N allele, and thereby concluding that the dog is predisposed to diabetes mellitus. The method may comprise determining that the genotype of the dog's KCNJ11 gene comprises one D274N allele and one reference allele, and thereby concluding that the dog is predisposed to diabetes mellitus. As set out above, a reference allele is a KCNJ11 allele that encodes wild-type canine Kir6.2 that does not comprise a D274N mutation. The reference allele may encode canine Kir6.2 of SEQ ID NO: 1. The reference allele does not comprise a C>T polymorphism in the canine KCNJ11 gene that encodes a D274N mutation in canine Kir6.2.

Heterozygosity for the D274N allele may lead to diabetes mellitus only in the presence of other diabetogenic factors. That is, heterozygosity for the D274N allele alone may not be sufficient to cause diabetes mellitus. Rather, further diabetogenic factors may be required to precipitate (i.e. lead to the onset of) diabetes mellitus in dogs heterozygous for the D274N allele. Accordingly, a KCNJ11 genotype heterozygous for the D274N allele may indicate that the dog is predisposed to diabetes mellitus when combined with one or more other diabetogenic factors. Therefore, the method may comprise determining that the genotype of the dog's KCNJ11 gene comprises one D274N allele, and thereby concluding that the dog is predisposed to diabetes mellitus when subject to one or more other diabetogenic factors. The method may comprise determining that the genotype of the dog's KCNJ11 gene comprises one D274N allele and one reference allele, and thereby concluding that the dog is predisposed to diabetes mellitus when subject to one or more other diabetogenic factors.

In the context of the present disclosure, a diabetogenic factor is a factor that contributes to the pathogenesis of diabetes mellitus. Numerous diabetogenic factors are known in the art. For instance, female sex is known to contribute to the pathogenesis of diabetes. In particular, the dioestrus stage of the canine oestrus cycle can lead to pancreatic beta cell stress due to levels of mammary gland derived growth hormone and luteal progesterone. Heterozygosity for the D274N allele may therefore confer a predisposition to diabetes mellitus in female dogs. This is demonstrated in the Example, which shows that there was a higher proportion of heterozygous females in the diabetic group than the control group.

Heterozygosity for the D274N allele may similarly confer a predisposition to diabetes mellitus in dogs subject to one or more other diabetogenic factors. Other diabetogenic factors may include, for example, obesity. Heterozygosity for the D274N allele may therefore confer a predisposition to diabetes mellitus in obese dogs.

Other diabetogenic factors may also include a medical condition, for example. For instance, pancreatitis or pancreatic stress may be a diabetogenic factor. Heterozygosity for the D274N allele may therefore confer a predisposition to diabetes mellitus in dogs having pancreatitis or pancreatic stress, or having a history of pancreatitis or pancreatic stress. Endocrine diseases may also be a diabetogenic factor. Heterozygosity for the D274N allele may therefore confer a predisposition to diabetes mellitus in dogs having an endocrine disease, or having a history an endocrine disease. Optionally, the endocrine disease is Cushing disease. Cushing's disease is a condition associated with high levels of blood cortisol, which can increase blood glucose and thus precipitate diabetes mellitus. Viral diseases may further be a diabetogenic factor. Heterozygosity for the D274N allele may therefore confer a predisposition to diabetes mellitus in dogs having a viral disease, or having a history of viral disease.

Other diabetogenic factors may also include treatment with certain drugs, for example. For instance, corticosteroid treatment may raise blood glucose and thus precipitate diabetes mellitus (in a similar way to Cushing's disease). Heterozygosity for the D274N allele may therefore confer a predisposition to diabetes mellitus in dogs treated with a corticosteroid, or having a history of corticosteroid treatment. Progestogens may cause pancreatic beta cells stress (e.g. by impairing insulin release and/or insulin sensitivity) and thus precipitate diabetes mellitus in a similar way to luteal progesterone in dioestrus. Heterozygosity for the D274N allele may therefore confer a predisposition to diabetes mellitus in dogs treated with a progestogen, or having a history of progestogen treatment.

Other diabetogenic factors may also include modifying genetic factors. For instance, the presence of a modifier locus may precipitate diabetes mellitus in a dog heterozygous for the D274N allele. In other words, heterozygosity for the D274N allele may confer a predisposition to diabetes mellitus in dogs having a particular genetic landscape or combination of traits.

Accordingly, a KCNJ11 genotype heterozygous for the D274N allele may indicate that the dog is predisposed to diabetes mellitus when it is subject to one or more other diabetogenic factors. The one or more other diabetogenic factors may selected from female sex, obesity, pancreatitis, pancreatic stress, progestogen treatment, corticosteroid treatment, endocrine disease (e.g. Cushing's disease), viral disease, and a modifying genetic factor, alone or in any combination.

In one aspect of the disclosure, the D274N allele may be determined to be absent from genotype of the dog's KCNJ11 gene. That is, the method may determine that the genotype of the dog's KCNJ11 gene does not comprise a D274N allele. In other words, the method may determine that the genotype of the dog's KCNJ11 gene comprises two reference alleles, i.e. that the genotype of the dog's KCNJ11 gene is homozygous for the reference allele. As set out above, a reference allele is a KCNJ11 allele that encodes wild-type canine Kir6.2 that does not comprise a D274N mutation. The reference allele may encode canine Kir6.2 of SEQ ID NO: 1. The reference allele does not comprise a C>T polymorphism in the canine KCNJ11 gene that encodes a D274N mutation in canine Kir6.2.

A KCNJ11 genotype homozygous for the reference allele may indicate that the dog is not predisposed to diabetes mellitus associated with a mutation in the KCNJ11 gene. Therefore, the method may comprise determining that the genotype of the dog's KCNJ11 gene comprises two reference alleles, and thereby concluding that the dog is not predisposed to KCNJ11-associated diabetes mellitus. It is though possible that a dog whose KCNJ11 genotype is homozygous for the reference allele may go on to develop diabetes mellitus. A dog whose KCNJ11 genotype is homozygous for the reference allele may, for example, develop diabetes mellitus as a result of environmental factors. A dog whose KCNJ11 genotype is homozygous for the reference allele may, for example, have one or more predispositions to diabetes mellitus that are not associated with KCNJ11. The one or more other predispositions may be associated with a mutation (e.g. a SNP) in one or more other genes other than KCNJ11.

### Determining Potential to Produce Predisposed Progeny

Disclosed herein is a method of determining the potential of a dog to produce progeny that are genetically predisposed to diabetes mellitus, comprising determining whether the genotype of the dog's KCNJ11 gene comprises a D274N allele. Determining the potential of a dog to produce progeny that are genetically predisposed to diabetes mellitus is advantageous, because dogs with such potential may be excluded from breeding programmes. In this way, the incidence of offspring having diabetes may be reduced.

### Dog

The method screens a dog for potential to produce progeny that are genetically predisposed to diabetes mellitus. The dog may be any canine individual, of any breed, age or sex. Any of the exemplary breeds, ages and sexes described above in connection with a method of screening a dog for a predisposition to diabetes mellitus may also apply to the method of determining the potential of a dog to produce progeny that are genetically predisposed to diabetes mellitus.

### Predisposition to Diabetes Mellitus

The purpose of the method is to identify the dog's potential to produce progeny with a predisposition to diabetes mellitus. Predisposition and diabetes mellitus are described above in connection with a method of screening a dog for a predisposition to diabetes mellitus. Any of the features or definitions above may also apply to the method of determining the potential of a dog to produce progeny that are genetically predisposed to diabetes mellitus.

### Determining Genotype

In order to determine the potential of the dog to produce progeny that are genetically predisposed to diabetes mellitus, the method comprises determining whether the genotype of the dog's KCNJ11 gene comprises a D274N allele. To determine whether the genotype of the dog's KCNJ11 gene comprises a D274N allele, the genotype of the KCNJ11 gene may be determined. The D274N allele, the corresponding KCNJ11 reference allele, and means for determining the genotype of the KCNJ11 gene are described above in connection with a method of screening a dog for a predisposition to diabetes mellitus. Any of the features or definitions above may also apply to the method of determining the potential of a dog to produce progeny that are genetically predisposed to diabetes mellitus.

### Link Between Genotype and Genetic Potential

Genotyping of the dog's KCNJ11 gene may determine that: (1) the dog's KCNJ11 gene comprises two D274N alleles (i.e. that the dog/gene is homozygous for the D274N allele); (2) the dog's KCNJ11 gene comprises one D274N allele and one reference allele (i.e. that the dog/gene is heterozygous for the D274N allele); or (3) the dog's KCNJ11 gene comprises two reference alleles (i.e. that the dog/gene is homozygous for the reference allele).

A KCNJ11 genotype homozygous or heterozygous for the D274N allele (in accordance with (1) or (2)) may indicate that the dog has potential to produce progeny that are genetically predisposed to diabetes mellitus. This is because the dog's genome comprises one or more D274N allele(s), and so at least some of the dog's gametes will comprise a D274N allele of KCNJ11. Specifically, all of the dog's gametes will comprise the D274N allele if the dog has a KCNJ11 genotype homozygous for the D274N allele, and around half of the dog's gametes will comprise the D274N allele if the dog has a KCNJ11 genotype heterozygous for the D274N allele. Therefore, progeny of the dog are capable of inheriting the D274N allele. As explained above, and demonstrated in the Example, dogs whose KCNJ11 gene comprises at least one D274N allele may be predisposed to diabetes mellitus. Specifically, D274N mutation of KCNJ11 may be a monogenic cause of diabetes mellitus in dogs whose KCNJ11 gene comprises two D274N alleles (i.e. in dogs homozygous for the D274N allele). D274N mutation of KCNJ11 may predispose to diabetes mellitus in dogs whose KCNJ11 gene comprises one D274N allele and one reference allele (i.e. in dogs heterozygous for the D274N allele) that are subject to other diabetogenic factors.

A KCNJ11 genotype homozygous for the KCNJ11 reference allele (in accordance with (3)) may indicate that the dog does not have potential to produce progeny that are genetically predisposed to diabetes mellitus associated with a mutation in the KCNJ11 gene. This is because the dog's genome does not comprise a D274N allele, and so the D274N allele will not be present in the dog's gametes. Therefore, progeny of the dog are incapable of inheriting the D274N allele or the associated predisposition to diabetes mellitus described above and in the Example. It is though possible that progeny of the dog whose KCNJ11 may go on to develop diabetes mellitus attributable to other factors, such as environmental factors and/or a mutation (e.g. a SNP) in one or more genes other than KCNJ11.

### Selecting a Treatment for Diabetes Mellitus

Disclosed herein is a method of selecting a treatment for diabetes mellitus in a dog, comprising determining whether the genotype of the dog's KCNJ11 gene comprises a D274N allele, and selecting a treatment based on the determined genotype.

Traditionally, canine diabetes has been treated with injectable insulin therapy, typically administered twice daily. Administration of insulin injections may be challenging for owners, and poorly tolerated by dogs. An oral treatment for canine diabetes is therefore desirable. As human neonatal diabetes caused by KCNJ11 mutations is treated by administering oral hypoglycaemic drugs, diabetic dogs possessing the D274N mutation in KCNJ11 may be treated in the same way. The selection method of the advantageously determines whether or not a given diabetic dog possesses the D274N mutation and, therefore, whether or not the dog is likely to benefit from treatment with an oral hypoglycaemic drug. Implementation of oral therapy in dogs likely to benefit from treatment with an oral hypoglycaemic drug spares the owner the inconvenience, and the dog the discomfort, of insulin injections. Furthermore, treatment with oral hypoglycaemic drug such sulphonylureas may reduce blood glucose fluctuations, lower HbA1c, and facilitate meal-stimulated insulin secretion.

### Dog

The method selects a treatment for diabetes mellitus in a dog. In other words, the method selects a treatment for a dog having diabetes mellitus. The dog is a diabetic dog.

The dog may be any canine individual, of any breed, age or sex. Any of the exemplary breeds, ages and sexes described above in connection with a method of screening a dog for a predisposition to diabetes mellitus may also apply to the method of selecting a treatment for diabetes mellitus in a dog.

### Diabetes Mellitus

The purpose of the method is to select a treatment for diabetes mellitus in a dog. Diabetes mellitus is described above in connection with a method of screening a dog for a predisposition to diabetes mellitus. Any of the features or definitions above may also apply to the method of selecting a treatment for diabetes mellitus in a dog.

### Determining Genotype

In order to select a treatment for diabetes mellitus in a dog, the method comprises determining whether the genotype of the dog's KCNJ11 gene comprises a D274N allele. To determine whether the genotype of the dog's KCNJ11 gene comprises a D274N allele, the genotype of the KCNJ11 gene may be determined. The D274N allele, the corresponding KCNJ11 reference allele, and means for determining the genotype of the KCNJ11 gene are described above in connection with a method of screening a dog for a predisposition to diabetes mellitus. Any of the features or definitions above may also apply to the method of selecting a treatment for diabetes mellitus in a dog.

### Selecting a Treatment

A treatment for diabetes mellitus is selected based on the determined genotype of the KCNJ11 gene. In particular, the treatment is selected based on whether or not the genotype of the KCNJ11 gene comprises one or more D274N allele.

The Example demonstrates that D274N-mutant K_{ATP} channels are less sensitive to ATP-mediated inhibition than wild-type K_{ATP} channels (i.e. K_{ATP} channels encoded by the KCNJ11 reference allele). In dogs having a KCNJ11 gene homozygous for the D274N allele, impaired insulin secretion associated with reduced K_{ATP} channel sensitivity may lead to the onset of diabetes. Impaired insulin secretion associated with reduced K_{ATP} channel sensitivity may also lead to the onset of diabetes in dogs having a KCNJ11 gene heterozygous for the D274N allele, for instance if the dog is subject to additional diabetogenic factors. Counteracting impaired insulin secretion associated with reduced K_{ATP} channel sensitivity may therefore be of therapeutic benefit in dogs whose KCNJ11 gene comprises one or more D274N allele (i.e. is homozygous or heterozygous for the D274N allele). An oral hypoglycaemic drug may, for example, be used to counteract impaired insulin secretion associated with reduced K_{ATP} channel sensitivity. Accordingly, an oral hypoglycaemic drug may be selected as the treatment if the genotype of the KCNJ11 gene comprises one or more D274N allele (i.e. if the KCNJ11 gene is homozygous or heterozygous for the D274N allele).

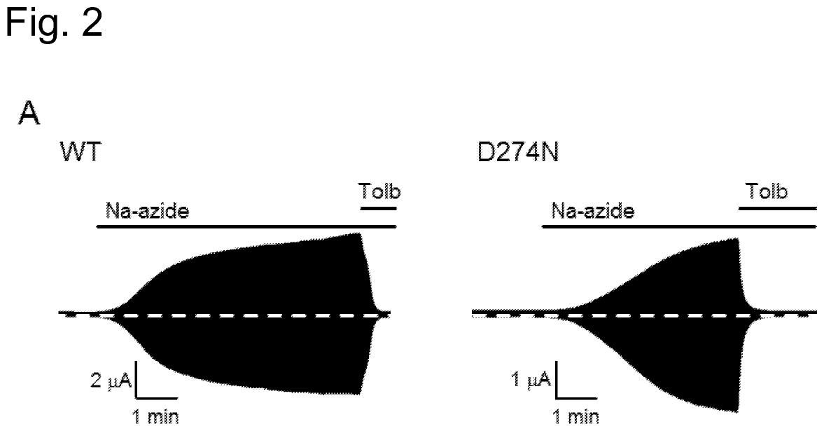

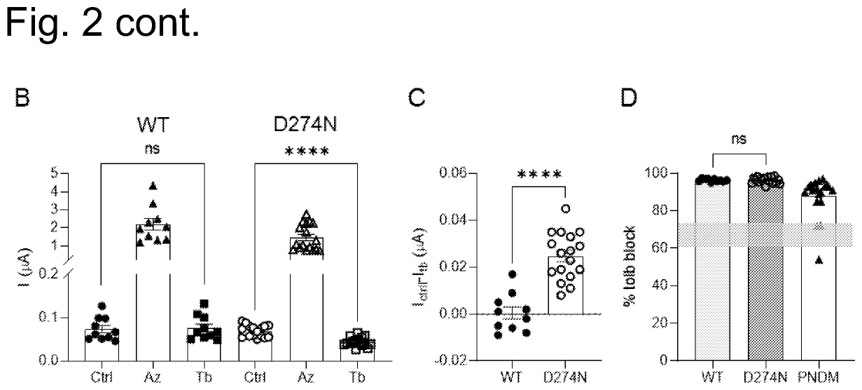

The oral hypoglycaemic drug may, for example, be capable binding to and/or closing a K_{ATP} channel. Such binding and closure may stimulate insulin secretion by pancreatic beta cells. Oral hypoglycaemic drugs capable of binding to and closing K_{ATP} channels are known in the art and include sulphonylureas. The Example confirms that sulphonylureas are capable of closing both wild-type and D274N K_{ATP} channels, achieving over 95% inhibition (see, for example, FIG. 2D). Accordingly, the oral hypoglycaemic drug may be a sulphonylurea. The sulphonylurea may, for example, be tolbutamide, glibenclamide or glipizide.

The quantity of oral hypoglycaemic drug to be administered, and the frequency of administration, depends on the dog to be treated. Precise amounts of oral hypoglycaemic drug to be administered, and the administration regimen, may depend on the judgement of the practitioner and may be peculiar to each subject.

When an oral hypoglycaemic drug is selected as the treatment, the selected treatment may further comprise injectable insulin. In other words, the selected treatment may comprise a combination of an oral hypoglycaemic drug and injectable insulin. Such combination therapy may, for example, reduce blood glucose fluctuations. The quantity of injectable insulin to be administered, and the frequency of administration, depends on the dog to be treated. Precise amounts of injectable insulin to be administered, and the administration regimen, may depend on the judgement of the practitioner and may be peculiar to each subject.

If the KCNJ11 gene is homozygous for the reference allele (i.e. does not comprise at least one D274N allele), the selected treatment may comprise injectable insulin. It may not be appropriate to administer an oral hypoglycaemic drug to a dog whose KCNJ11 gene is homozygous for the reference allele because K_{ATP} channel sensitivity may not be reduced. Administration of an oral hypoglycaemic drug may not, therefore, have the effect of counteracting K_{ATP} channel sensitivity so may not stimulate insulin secretion. Insulin supplementation may therefore be required.

### Prevention, Delay or Treatment of Diabetes Mellitus

Disclosed herein is a method of preventing, delaying or treating diabetes mellitus in a dog whose KCNJ11 gene comprises one or more D274N alleles, comprising administering an oral hypoglycaemic drug to the dog. Also disclosed herein is a composition comprising an oral hypoglycaemic drug for use in a method of preventing, delaying or treating diabetes mellitus in a dog whose KCNJ11 gene comprises one or more D274N alleles, wherein the method comprises administering the oral hypoglycaemic drug to the dog.

Further disclosed herein is a method of preventing or delaying diabetes mellitus in a dog whose KCNJ11 gene comprises one or more D274N alleles, comprising: (a) providing the dog with an anti-hyperglycaemic diet; or (b) neutering the dog, wherein the dog is female. Also disclosed herein is an anti-hyperglycaemic diet for use in a method of preventing or delaying diabetes mellitus in a dog whose KCNJ11 gene comprises one or more D274N alleles.

As set out above, human neonatal diabetes caused by KCNJ11 mutations is treated by administering oral hypoglycaemic drugs, such as sulphonylureas. The Example of the present disclosure identifies the D274N mutation of the canine KCNJ11 gene as a monogenic cause of diabetes mellitus in the dog. The Example also demonstrates that D274N-mutant K_{ATP} channels are less sensitive to ATP-mediated inhibition than wild-type K_{ATP} channels (i.e. K_{ATP} channels encoded by the KCNJ11 reference allele). Such reduced sensitivity may lead to impaired insulin secretion in dogs whose KCNJ11 gene comprises one or more D274N alleles. The Example further demonstrates that oral hypoglycaemic drugs, such as tolbutamide (a sulphonylurea), inhibits D274N-mutant K_{ATP} channels by more than 95%. Plausibly, therefore, oral hypoglycaemic drugs used to treat human neonatal diabetes caused by KCNJ11 mutations may also be used to treat diabetes mellitus associated with D274N mutation of the canine KCNJ11 gene. Such oral treatment is advantageous relative to traditional twice-daily injectable insulin therapy, as oral drugs are simpler to administer and may be better tolerated by the dog. In addition, treatment with oral hypoglycaemic drugs may reduce blood glucose fluctuations, lower HbA1c, and facilitate meal-stimulated insulin secretion. The present disclosure therefore provides a beneficial new treatment regimen for canine diabetes.

The present disclosure also provides means for preventing or delaying diabetes mellitus in a dog whose KCNJ11 gene comprises one or more D274N alleles. As explained above, the presence of one or more D274N alleles may indicate that the dog is predisposed to diabetes mellitus. Screening dogs for the presence of one or more D274N alleles can therefore identify dogs at risk of developing diabetes mellitus. In these dogs, prophylactic measures may be implemented before the development of clinical disease (i.e. in the pre-diabetic state) to delay or prevent the onset of diabetes mellitus. For instance, steps may be taken to minimise the occurrence of hyperglycaemia in the dog.

Hyperglycemia is toxic to insulin-producing pancreatic beta cells, and so minimising the occurrence of hyperglycemia may help to preserve beta cells and/or their ability to produce insulin. The occurrence of hyperglycemia may be minimised by (1) administering an oral hypoglycaemic drug, (2) providing an anti-hyperglycaemic diet, and/or (3) neutering female dogs. The occurrence of hyperglycemia may be minimised by: (1); (2); (3); (1) and (2); (1) and (3); (2) and (3); or (1), (2) and (3).

### Dog

The method comprises administering an oral hypoglycaemic drug to a dog to prevent, delay or treat diabetes mellitus, or providing an anti-hyperglycaemic diet to a dog to prevent or delay diabetes mellitus. The dog is a dog whose KCNJ11 gene comprises one or more D274N alleles. The KCNJ11 gene may, for example, comprise one D274N allele and one reference allele. In other words, the KCNJ11 gene may be heterozygous for the D274N allele. The KCNJ11 gene may, for example, comprise two D274N alleles. In other words, the KCNJ11 gene may be homozygous for the D274N allele.

As explained above, dogs whose KCNJ11 gene comprises one or more D274N alleles may be predisposed to diabetes mellitus. Therefore, the dog may be a dog that is predisposed to diabetes mellitus. The dog may be predisposed to diabetes mellitus based on KCNJ11 genotype alone. For instance, a KCNJ11 genotype homozygous for the D274N allele may be sufficient to confer a predisposition for diabetes mellitus. The dog may be predisposed to diabetes mellitus when its KCNJ11 genotype is combined with other diabetogenic factors, such as those mentioned above. For instance, a KCNJ11 genotype heterozygous for the D274N allele may lead to a predisposition for diabetes mellitus when combined with other diabetogenic factors such as female sex, obesity, pancreatitis, pancreatic stress, progestogen treatment, corticosteroid treatment, endocrine disease, viral disease, or a modifying genetic factor.

The dog predisposed to diabetes mellitus, may not (or may not yet) have diabetes mellitus. In this case, the method provides prophylactic treatment against the development of diabetes mellitus. That is, the method prevents or delays the development of diabetes mellitus. A dog that is predisposed to diabetes mellitus but which does not have diabetes mellitus may be in a pre-diabetic state. Therefore, the dog to be treated prophylactically may be in a pre-diabetic state. The dog to be treated prophylactically may have pre-diabetes. A dog that is in a pre-diabetic state, or that has pre-diabetes, may have impaired glucose tolerance which may result in inappropriately high blood glucose following a meal or following ingestion or injection of glucose. A fasting blood glucose level of between 7 mmol/l and 11 mmol/l may, for example, be indicative of pre-diabetes in dogs. Treatment of a dog that is in a pre-diabetic state may halt progression of the pre-diabetic state to clinical or symptomatic diabetes mellitus. Treatment of a dog that is in a pre-diabetic state may for example improve glucose tolerance, such that blood glucose is not inappropriately high following a meal or following ingestion or injection of glucose. Treatment of a dog that is in a pre-diabetic state may for example restore fasting blood glucose to below 7 mmol/l.

The dog whose KCNJ11 gene comprises one or more D274N alleles may be a diabetic dog. In other words, dog whose KCNJ11 gene comprises one or more D274N alleles may have diabetes mellitus. That is, the dog may already have clinical disease (rather than being merely predisposed to diabetes mellitus). In this case, the method provides therapeutic treatment for diabetes mellitus. That is, the method treats the diabetes mellitus. Treatment of diabetes mellitus alleviates the clinical signs of diabetes mentioned above. Clinical signs are usually only evident when blood glucose is above the renal threshold for glucose reabsorption (around 12 mmol/l). Therefore, treatment of diabetes mellitus may restore the blood glucose level to 12 mmol/l or less.

The dog may be any canine individual, of any breed, age or sex. Any of the exemplary breeds, ages and sexes described above in connection with a method of screening a dog for a predisposition to diabetes mellitus may also apply to the method of selecting a treatment for diabetes mellitus in a dog.

### Diabetes Mellitus

The purpose of the method is to prevent, delay or treat diabetes mellitus in a dog whose KCNJ11 gene comprises one or more D274N alleles. Diabetes mellitus is described above in connection with a method of screening a dog for a predisposition to diabetes mellitus. Any of the features or definitions above may also apply to aspects of the disclosure concerning prevention, delay or treatment of diabetes mellitus.

### Administering an Oral Hypoglycaemic Drug

The method may comprise administering an oral hypoglycaemic drug to the dog, in order to prevent, delay or treat diabetes mellitus. The oral hypoglycaemic drug may be any drug that can be administered orally in order to reduce blood glucose level. The oral hypoglycaemic drug may, for example, be capable binding to and/or closing a K_{ATP} channel. Such binding and closure may stimulate insulin secretion by pancreatic beta cells, thereby reducing blood glucose level. Oral hypoglycaemic drugs capable of binding to and closing K_{ATP} channels are known in the art and include sulphonylureas. Accordingly, the oral hypoglycaemic drug may be a sulphonylurea. The sulphonylurea may, for example, be tolbutamide, glibenclamide or glipizide.

The quantity of oral hypoglycaemic drug to be administered, and the frequency of administration, may depends on the dog to be treated. Precise amounts of oral hypoglycaemic drug to be administered, and the administration regimen, may depend on the judgement of the practitioner and may be peculiar to each subject. Typically, the oral hypoglycaemic drug is administered once daily or twice daily.

Administration of the oral hypoglycaemic drug may prevent diabetes mellitus. In other words, administration of the oral hypoglycaemic drug may stop diabetes mellitus from developing in the dog. In this way, a dog predisposed to diabetes mellitus may be protected from clinical disease indefinitely.

Administration of the oral hypoglycaemic drug may delay diabetes mellitus. In other words, administration of the oral hypoglycaemic drug may lengthen the time to onset of diabetes mellitus. In this way, a dog predisposed to diabetes mellitus may be afforded a longer period free from clinical disease. The onset of diabetes may, for example, be delayed by one or more months, such as two or more, three or more, four or more, five or more, six or more, seven or more, eight or more, nine or more, ten or more, eleven or more, or twelve or more months. The onset of diabetes may, for example, be delayed by one to twelve months, two to eleven months, three to ten months, four to nine months, five to eight months, or six to seven months. The onset of diabetes may, for example, be delayed by one or more years, such as two or more, three or more, four or more, five or more, six or more, seven or more, eight or more, nine or more, ten or more, eleven or more, twelve or more, thirteen or more, fourteen or more, or fifteen or more years. The onset of diabetes may, for example, be delayed by one to fifteen years, two to fourteen years, three to thirteen years, four to twelve years, five to eleven years, six to ten years, or seven to nine years.

Administration of the oral hypoglycaemic drug may treat diabetes mellitus. In other words, administration of the oral hypoglycaemic drug may alleviate the signs or symptoms of diabetes mellitus in dog having clinical disease. Typically, administration of the oral hypoglycaemic drug lowers the blood glucose level in the dog.

The method may further comprise providing one or more other therapies to the dog. For example, the method may comprise providing the dog with an anti-hyperglycaemic diet. Anti-hyperglycaemic diets are described in more detail below. The method may further comprise administering the dog with injectable insulin. The method may further comprise providing the dog with an anti-hyperglycaemic diet and administering the dog with injectable insulin. Typically, injectable insulin therapy is reserved for dogs already having diabetes (rather than dogs predisposed to diabetes), in which it provides a therapeutic effect. Combination therapy with an oral hypoglycaemic drug and injectable insulin may, for example, reduce blood glucose fluctuations. The quantity of injectable insulin to be administered, and the frequency of administration, depends on the dog to be treated. Precise amounts of injectable insulin to be administered, and the administration regimen, may depend on the judgement of the practitioner and may be peculiar to each subject.

The method may further comprise providing one or more other therapies to the dog. For example, the method may comprise (i) providing the dog with an anti-hyperglycaemic diet, and/or (ii) neutering the dog when the dog is female. Anti-hyperglycaemic diets and neutering are described below.

### Administering an Anti-Hyperglycaemic Diet

The method may comprise providing an anti-hyperglycaemic diet to the dog, in order to prevent or delay diabetes mellitus. The nature of KCNJ11-associated diabetes mellitus in the dog is such that dietary management alone is unlikely to be successful. However, dietary management of dogs predisposed to KCNJ11-associated diabetes mellitus (i.e. dogs whose KCNJ11 gene comprises one or more D274N alleles) may guard against progression to clinical disease.

An anti-hyperglycaemic diet may be defined as a diet that minimises post-prandial elevations in blood glucose. In other words, an anti-hyperglycaemic diet may stabilise blood glucose levels, or limit spikes and dips in blood-glucose levels. Anti-hyperglycaemic diets are known in the art and may, for example, be formulated to have a low glycaemic index (GI). An anti-hyperglycaemic diet may, for example, be (a) low in carbohydrate, (b) high in protein, and/or (c) high in soluble fibre. An anti-hyperglycaemic diet may, for example, be: (a); (b); (c); (a) and (b); (a) and (c); (b) and (c); or (a), (b) and (c). An anti-hyperglycaemic diet may comprise complex carbohydrates. An anti-hyperglycaemic diet may lack (or substantially lack) simple carbohydrates. An anti-hyperglycaemic diet may comprise complex carbohydrates and lack (or substantially lack) simple carbohydrates. An anti-hyperglycaemic diet may comprise more complex carbohydrates than simple carbohydrates.

The anti-hyperglycaemic diet may, for example, be a complete dog food. A complete dog food is a food that provides all the nutrients in the amount and proportion that a dog needs. A dog fed a complete dog food need not be fed any other foods in order to meet its nutritional requirements. The anti-hyperglycaemic diet may therefore be formulated to be fed to the dog as its exclusive diet. The anti-hyperglycaemic diet may be a dry dog food or a wet dog food.

Provision of the anti-hyperglycaemic diet may prevent diabetes mellitus. In other words, provision of the anti-hyperglycaemic diet may stop diabetes mellitus from developing in the dog. In this way, a dog predisposed to diabetes mellitus may be protected from clinical disease indefinitely.

Provision of the anti-hyperglycaemic diet may delay diabetes mellitus. In other words, provision of the anti-hyperglycaemic diet may lengthen the time to onset of diabetes mellitus. In this way, a dog predisposed to diabetes mellitus may be afforded a longer period free from clinical disease. The onset of diabetes may, for example, be delayed by one or more months, such as two or more, three or more, four or more, five or more, six or more, seven or more, eight or more, nine or more, ten or more, eleven or more, or twelve or more months. The onset of diabetes may, for example, be delayed by one to twelve months, two to eleven months, three to ten months, four to nine months, five to eight months, or six to seven months. The onset of diabetes may, for example, be delayed by one or more years, such as two or more, three or more, four or more, five or more, six or more, seven or more, eight or more, nine or more, ten or more, eleven or more, twelve or more, thirteen or more, fourteen or more, or fifteen or more years. The onset of diabetes may, for example, be delayed by one to fifteen years, two to fourteen years, three to thirteen years, four to twelve years, five to eleven years, six to ten years, or seven to nine years.

The method may further comprise providing one or more other therapies to the dog. For example, the method may comprise (i) providing the dog with an oral hypoglycaemic drug, and/or (ii) neutering the dog before the first oestrus cycle, when the dog is female. Suitable oral hypoglycaemic drugs are described above. The quantity of the oral hypoglycaemic drug to be administered, and the frequency of administration, depends on the dog to be treated. Precise amounts of oral hypoglycaemic drug to be administered, and the administration regimen, may depend on the judgement of the practitioner and may be peculiar to each subject. Neutering before the first oestrus cycle is described below.

### Neutering Before the First Oestrus Cycle

When the dog is female, the method may comprise neutering the dog in order to prevent or delay diabetes mellitus. Oestrus can cause hyperglycemia through the insulin-antagonistic effects of progesterone and growth hormone during the dioestrus phase. Neutering minimises such hormonal effects and thus guards against hyperglycemia.

Neutering of a female dog may refer to ovariohysterectomy or ovariectomy. Therefore, the method may comprise performing an ovariohysterectomy or ovariectomy on the dog in order to prevent or delay diabetes mellitus.

The effect of neutering on minimising the hormonal effects mentioned above may be greater the earlier that neutering is performed. Therefore, neutering may preferably be performed before the dog's first oestrus cycle. Dogs typically have their first oestrus cycle at 6 to 12 months of age, and the signs of oestrus are well-recognised. Therefore, for a dog of known history, it should be within the skilled person's routine skill set to determine whether or not the dog has had its first oestrus cycle. If the history of the dog is not known (for instance, if the dog is a rescue dog), then neutering may preferably be performed at the earliest practical opportunity. Neutering may also be performed at the earliest practical opportunity if the first oestrus cycle has already passed, for instance if the dog did not present until after its first oestrus cycles or there were medical reasons as to why neutering before the first oestrus cycle was not possible.

Neutering may prevent diabetes mellitus. In other words, neutering may stop diabetes mellitus from developing in the dog. In this way, a dog predisposed to diabetes mellitus may be protected from clinical disease indefinitely.

Neutering may delay diabetes mellitus. In other words, neutering may lengthen the time to onset of diabetes mellitus. In this way, a dog predisposed to diabetes mellitus may be afforded a longer period free from clinical disease. The onset of diabetes may, for example, be delayed by one or more months, such as two or more, three or more, four or more, five or more, six or more, seven or more, eight or more, nine or more, ten or more, eleven or more, or twelve or more months. The onset of diabetes may, for example, be delayed by one to twelve months, two to eleven months, three to ten months, four to nine months, five to eight months, or six to seven months. The onset of diabetes may, for example, be delayed by one or more years, such as two or more, three or more, four or more, five or more, six or more, seven or more, eight or more, nine or more, ten or more, eleven or more, twelve or more, thirteen or more, fourteen or more, or fifteen or more years. The onset of diabetes may, for example, be delayed by one to fifteen years, two to fourteen years, three to thirteen years, four to twelve years, five to eleven years, six to ten years, or seven to nine years.

The method may further comprise providing one or more other therapies to the dog. For example, the method may comprise providing the dog with (i) an oral hypoglycaemic drug and/or (ii) an anti-hyperglycaemic diet. Suitable oral hypoglycaemic drugs are described above. The quantity of the oral hypoglycaemic drug to be administered, and the frequency of administration, depends on the dog to be treated. Precise amounts of oral hypoglycaemic drug to be administered, and the administration regimen, may depend on the judgement of the practitioner and may be peculiar to each subject. Anti-hyperglycaemic diets are also described above.

The following Examples illustrate the invention.

### Example 1

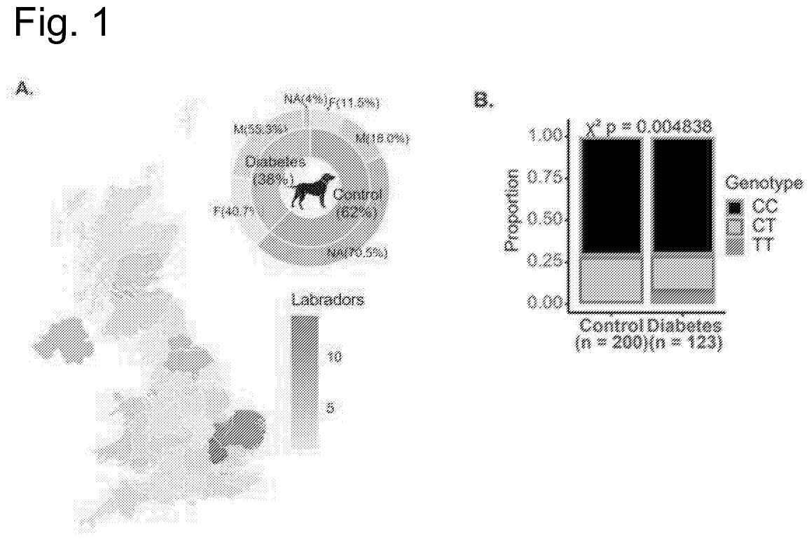

A homozygous non-synonymous variant rs851344999 in KCNJ11 was identified in two diabetic individuals during exome sequencing analysis of 4 Labrador retrievers with diabetes onset at <6 months of age and 1 dog with diabetes onset in adulthood. The presence of the alternate T allele was predicted to cause a D274N amino acid change in the canine Kir.2 protein. Subsequent genotyping of a further 117 diabetic Labrador retrievers and Labrador retriever crosses from the UK Canine Diabetes Database and Archive, using a range of techniques, identified a further 5 diabetic individuals homozygous for the same mutation and 28 heterozygotes (FIG. 1).

Genotyping of a total of 200 adult Labrador retrievers with no reported clinical signs of diabetes mellitus identified only one dog that was homozygous for the D274N mutation and 56 heterozygotes. The association between rs851344999 genotype and diabetes in the Labrador retriever was significant, with a likely recessive monogenic inheritance (χ² p=0.004838). Clinical records suggested that the homozygote control dog had suffered from pancreatitis an adulthood but did not record a diagnosis of diabetes prior to euthanasia at the age of 10 years, although a blood glucose measurement had not been recorded at that time. The finding of one control dog that was homozygous for the diabetes-associated variant without a diabetes phenotype being reported at the time of sampling could be the result of very late onset or unrecognized diabetes mellitus in this patient.

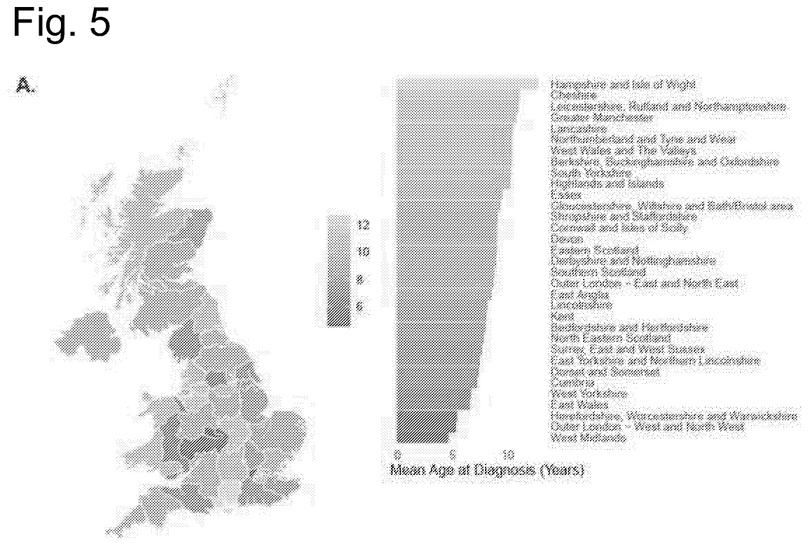

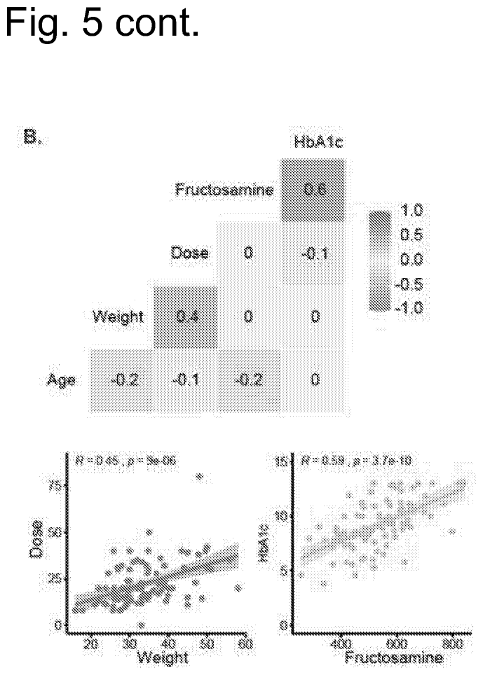

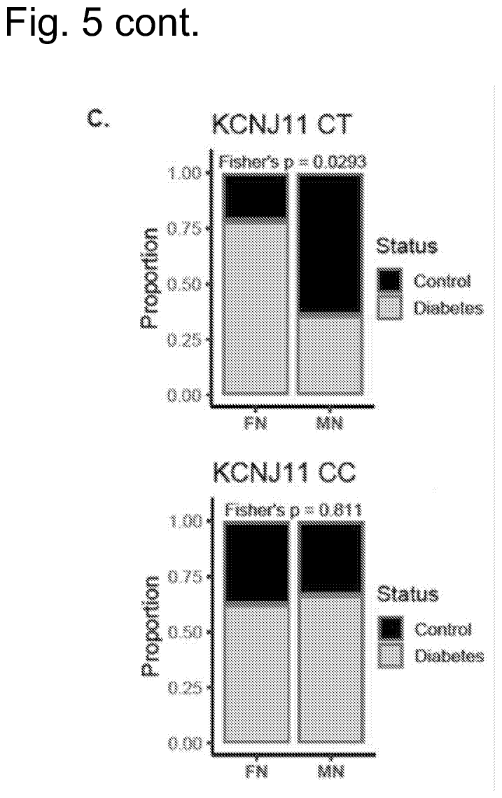

The samples from the juvenile onset diabetes dogs in the study (n=13) were mainly submitted by veterinary practices clustered in the Herefordshire, Worcestershire, Warwickshire and the West Midlands. Where signalment and clinical metadata was available for the diabetic dogs, there was no association between the presence or absence of the D274N mutation and age of onset of diabetes. Amongst the diabetic Labrador retrievers, the proportion of neutered females that were homozygous for the reference allele of KCNJ11 rs851344999 (CC) was not significantly different than the proportion of homozygous neutered males. However, there was a significant difference in the proportion of neutered female KCNJ11 rs851344999 heterozygotes (CT) compared to neutered male heterozygotes in the diabetic Labrador retriever population (Fisher's p=0.0293) (FIG. 5).

Given the significant association between rs851344999 genotype and diabetes mellitus in the Labrador retriever (χ² p=0.004838), and the potential monogenic recessive impact of the variant on diabetes phenotype and beta-cell function, we undertook functional studies of the D274N mutation. First, we examined the effect of the Kir6.2-D274N mutation on the metabolic regulation of the K_{ATP} channel by measuring whole-cell currents in Xenopus oocytes. In control solution, wild-type (WT) channels are closed due to the high intracellular concentration of adenosine triphosphate ([ATP]ᵢ), but they can be activated by lowering [ATP]; with the metabolic inhibitor Na-azide (FIGS. 2A and B). Homozygous Kir6.2-D274N/SUR1 currents (D274N) were similarly activated. The K_{ATP} channel inhibitor tolbutamide (0.5 mM, also used as an oral sulfonylurea hypoglycemic agent) blocked both WT and D274N channels by >95% (FIG. 2D). WT currents were of similar magnitude in control and tolbutamide solution, indicating that WT channels are closed at resting [ATP]; in oocytes (FIGS. 2B and C). In contrast, D274N currents were significantly smaller when tolbutamide was present, suggesting that D274N channels are less blocked by resting [ATP]ᵢ. In beta-cells, this would be expected to reduce glucose-stimulated K_{ATP} channel closure and insulin secretion, thereby accounting for the dogs' diabetes.

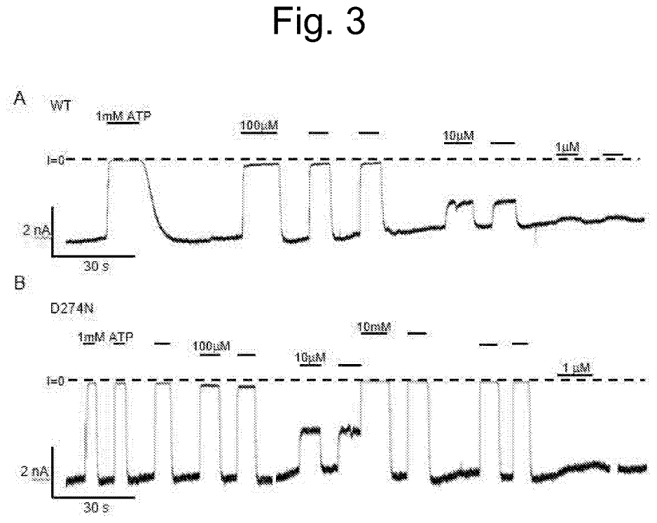

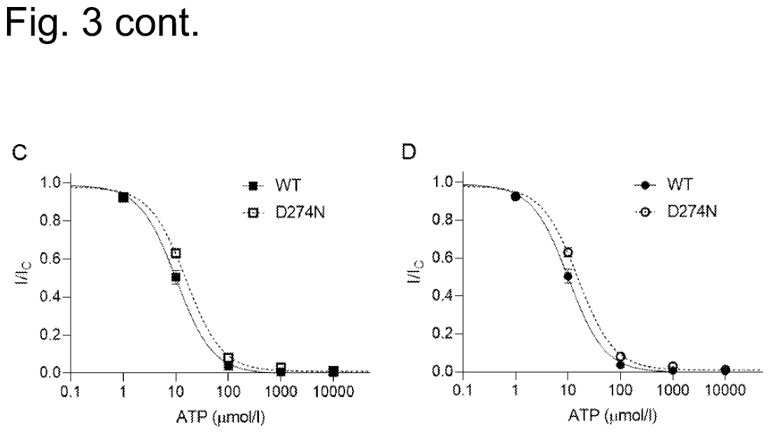

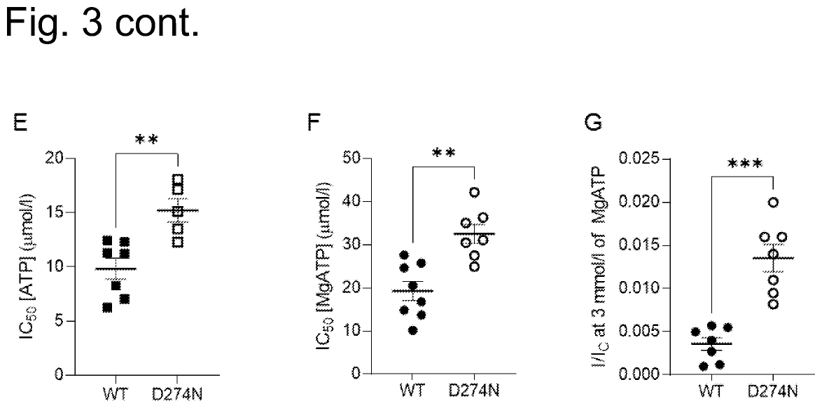

We measured the ATP sensitivity of WT and D274N channels in both the absence and presence of Mg²⁺ (FIGS. 3A and B). The former (absence of Mg²⁺) isolates ATP binding at Kir6.2, the latter (presence of Mg²⁺) more closely approximates the physiological condition. The ATP concentration that produced half-maximal current inhibition was slightly, but significantly, increased in D274N channels both in the absence of Mg^{2±} (WT, IC₅₀=10±1 μmol/l, n=7; D274N, IC₅₀=15±1 μmol/l, n=5; p=0.0044; FIGS. 3C and E) and in its presence (WT, IC₅₀=19±2 μmol/l, n=8; D274N, IC₅₀=32±2 μmol/l, n=7; p=0.0011; FIGS. 3D and F). Additionally, the percentage of unblocked current at 3 mmol/l MgATP was significantly increased (FIG. 3G). Thus D274N channels are slightly less sensitive to ATP inhibition.

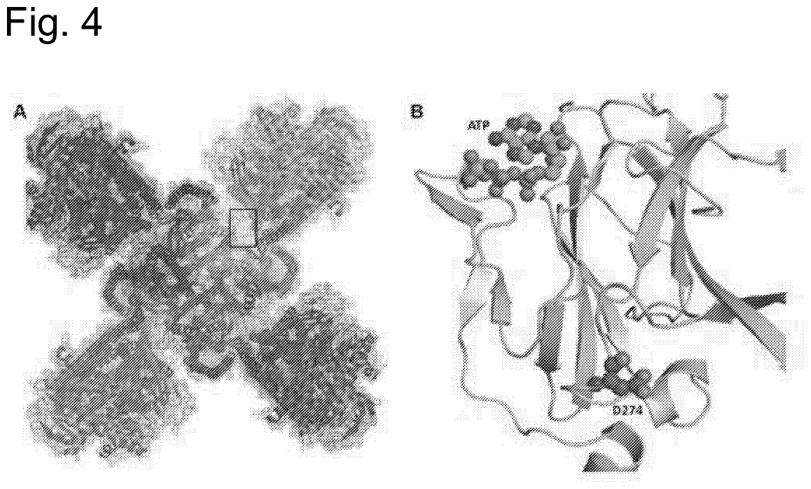

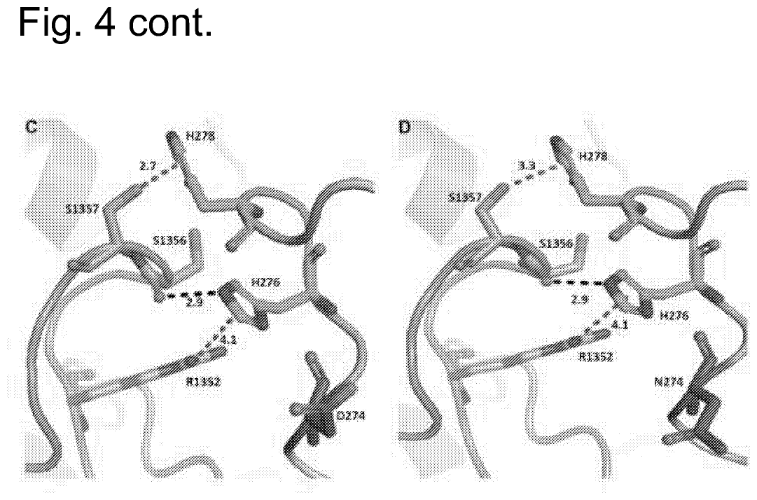

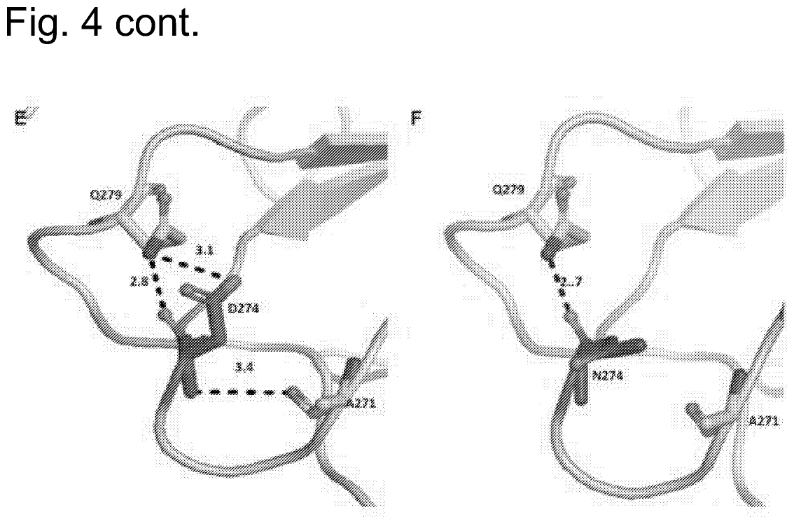

The inhibitory ATP-binding site of the K_{ATP} channel lies at the interface between two adjacent Kir6.2 subunits and also has contributions from SUR1. As D274 lies far from this site (FIG. 4), it is unlikely that the D274N mutation impairs ATP binding directly. Nevertheless, because D274 is highly conserved, it may play an important structural and/or functional role and its mutation could reduce ATP binding indirectly. For example, it might allosterically impair ATP binding, enhance the intrinsic open probability of the channel or impair the mechanism by which ATP binding is transduced into closure of the channel gate. Interestingly, D274 is followed by a triple histidine motif, that has been suggested as a potential polar contact between Kir6.2 and SUR1-NBD2. Thus, it is possible that the D274N mutation influences ATP binding by perturbing the interaction between Kir6.2 and SUR1.

The shift in ATP sensitivity produced by D274N mutation is relatively small, but consistent with the finding that tiny differences in ATP sensitivity can cause neonatal diabetes in humans. The magnitude of the shift produced by D274N mutation is similar in magnitude to differences in ATP sensitivity causing neonatal diabetes in humans. This is unsurprising as small changes in current amplitude translate into large changes in beta-cell membrane potential and electrical activity and thereby in insulin secretion. The small magnitude of the shift may explain why heterozygosity did not appear to be associated with increased diabetes risk, with carriers only developing diabetes if they carry an additional genetic load or lifestyle factors. Notably there was a higher proportion of heterozygous females in the diabetic group than the control group, suggesting that in certain circumstances heterozygosity may contribute to disease risk. The unique diabetogenic state of the dioestrus phase of the oestrus cycle in female dogs, related to mammary gland derived growth hormone, as well as luteal progesterone, may contribute to pancreatic beta cell stress. As the information about the number of oestrus cycles occurring in each female prior to neutering is not available, it was not possible to explore this further within the dataset.

The impact of the D274N mutation on the structure of the K_{ATP} channel was modelled (FIG. 4). The inhibitory ATP-binding site of the K_{ATP} channel lies at the interface between two adjacent Kir6.2 subunits with contributions from SUR1. D274 is highly conserved across species but lies far from this site, so the D274N mutation is unlikely to alter ATP binding directly. However, it may have an indirect effect, allosterically impairing ATP binding, enhancing the probability of the channel being open or impairing the mechanism by which ATP binding is transduced into closure of the channel gate. Interestingly, D274 is followed by a triple histidine motif, that has been suggested as a potential polar contact between Kir6.2 and SUR1-NBD2. Thus, the D274N mutation may influence ATP binding by perturbing the interaction between Kir6.2 and SUR1 (FIG. 4).

Sulphonylurea drugs are routinely used to treat human diabetes because they bind to the SUR1 subunit of the K_{ATP} channel and cause it to close, thereby stimulating insulin secretion. The sulphonylurea tolbutamide inhibited both WT and D274N channels by >95% (FIG. 2D). Human neonatal diabetes patients carrying Kir6.2 mutations that are inhibited to this extent can be successfully treated with oral sulphonylureas, provided drug therapy is commenced early after diagnosis. Therefore, oral sulphonylureas may rationally be an effective therapy for canine diabetes associated with the D274N mutation.

Twice-daily injectable insulin therapy is the mainstay of treatment for diabetes in dogs. However, oral drugs are simpler to administer for the owner and potentially better tolerated by the dog. Furthermore, in humans with KCNJ11 mutations there are clear clinical benefits to early treatment with oral sulphonylureas: they reduce blood glucose fluctuations, lower HbA1c and facilitate meal-stimulated insulin secretion (by enabling incretin action), and in some cases are used in combination with insulin. Similar benefits may be expected in dogs.

Genetic screening of diabetic Labrador retrievers for KCNJ11 mutations could identify heterozygous and homozygous animals that may be suitable for oral drug therapy as part of a precision medicine approach to canine diabetes management. Genotyping would also enable informed avoidance of inter-breeding of carrier Labrador retrievers within the breeding pool and thereby potentially reduce the incidence of diabetes in the breed.

### Materials and Methods

### Molecular Genetics

### Animals

All blood samples used in this study were collected for a clinical veterinary purpose and the residual samples available for study were surplus to clinical requirements. Samples were archived and utilised with the permission of the Royal Veterinary College Clinical Research Ethical Review Board (URN 2017 1685-3) and with the consent of the owner of the animal.

All diabetic Labrador retriever and Labrador retriever cross blood samples were obtained from the UK Canine Diabetes Database and Archive, established in 1999 at the Royal Veterinary College. Diabetes was diagnosed based on the presence of appropriate clinical signs of polyuria and polydipsia, and the presence of persistent hyperglycemia. Where available, data were recorded regarding age, breed, sex, neutering status, insulin dose and body weight at time of sample submission. Measurement of serum fructosamine and HbA1c was also undertaken on submitted samples for clinical monitoring purposes. Control (non-diabetic) Labrador retriever samples were obtained from the Clinical Investigation Centre research archives, established at the Royal Veterinary College and containing residual samples from patients being treated at the Queen Mother Hospital for Animals. An additional cohort of non-diabetic control Labrador retriever DNA was provided by Dr Catheryn Mellersh, University of Cambridge, following completion of a different study at the Animal Health Trust, UK.

The control group was age-restricted, to minimise the possibility of controls developing diabetes mellitus in later life. All control Labrador retrievers had reached a minimum age of 7 years without clinical signs of diabetes mellitus.

### DNA Extraction and Purification

DNA was extracted from archived residual EDTA blood, blood clot or buffy coat using Qiagen DNEasy Blood and Tissue kit, according to manufacturer's instructions, and including and RNAse treatment step. Further purification and concentration was performed with a DNA Clean and Concentrator kit (Zymo Research). The concentration of DNA in 1 μL of eluate was measured using a spectrophotometer (Nanodrop) and DNA integrity was assessed using agarose gel electrophoresis and/or TapeStation (Agilent Technologies, Inc).

### Variant Discovery

For variant discovery, canine exome sequencing was undertaken on 10 dogs at Otogenetics (Atlanta, USA) using DNA extracted from archived blood samples. Data were analysed using a bespoke bioinformatics pipeline based on Genome Analysis Toolkit best practices (GATK3) on a high performance computer cluster (BMRC Cluster, Wellcome Centre for Human Genetics, University of Oxford). Variant effect prediction was carried out using SNPEff, and a candidate gene approach was taken to identify non-synonymous variants present in diabetic dogs and located in genes known to be associated with monogenic diabetes in humans.

### Follow Up Genotyping

Genotypes at the KNCJ11 variant were ascertained in further Labrador retrievers by a range of technologies. As well as undertaking targeted PCR and TaqMan sequencing specifically for this study, genotypes from diabetic and control dogs were available within unpublished high throughput sequencing datasets generated by the authors for other projects.

In addition, a custom TaqMan genotyping assay was used for genotyping individual samples. Illumina Whole Genome Sequencing data was undertaken at Edinburgh Genomics using a HiSeqX sequencer with 150 bp paired end reads at 30× coverage. Targeted genotyping data were also available from two different custom high throughput sequencing based panels which both captured of the variant of interest. The first was TWIST Bioscience Target Enrichment (Twist Bioscience, CA, USA) and the second was SeqSNP Targeted Genotyping (LGC Genomics, Teddington, UK).

### External Reference Data Sources

Additional external data sources were examined to determine the alternate allele frequency of the KCNJ11 variant of interest in other populations. These comprised unpublished data from the ‘Give a Dog a Genome’ project, as well as publicly available data from The NHGR Dog Genome Project (https://research.nhgri.nih.gov/dog_genome/) and the Dog BioMedical Variant Database Consortium.

### Mutagenesis and Expression

Human Kir6.2 and SUR1 were subcloned into pcDNA4/TO or pBF expression vectors. Site-directed mutagenesis was performed using the QuikChange XL system (Stratagene; San Diego, CA), and verified by sequencing (DNA Sequencing and Services; Dundee, Scotland). Preparation of mRNA was performed as described (2). HEK-293T cells were maintained in Dulbecco's Modified Eagle Medium (DMEM, Sigma), supplemented with 10% (v/v) fetal bovine serum (Life Technologies Ltd), 100 U/mL penicillin, and 100 μg/mL streptomycin (Thermo Fisher Scientific; Waltham, MA) at 37° C., 5% CO₂/95% air. When close to confluency, cells were trypsinised and seeded in 25 cm² flasks. After 3 h, cells were transfected with wild-type (WT) SUR1 and WT or mutant Kir6.2, using TransIT-LT1 (Mirus Bio LLC). Cells were re-plated onto poly-L-lysine treated 35 mm petri dishes (Corning) 48 hours post-transfection. Measurements were performed 72 hours post-transfection.

Xenopus oocytes were prepared as previously described (Gribble et al, 1997), injected with 0.8 ng wild-type or mutant Kir6.2 mRNA and ~4 ng SUR1 mRNA, and maintained in Barth's solution (in mM: 88 NaCl, 1 KCl, 1.68 MgSO₄, 0.41 CaCl₂), 0.47 Ca(NO₃)₂, 2.4 NaHCO₃, 10 HEPES, adjusted to pH 7.4 with NaOH) at 18° C. Currents were recorded 2-4 days after injection.

### Electrophysiology

### Two-Electrode Voltage-Clamp Recordings

Whole-cell WT or mutant K_{ATP} currents were recorded at 22-24° C. in response to voltage steps of ±20 mV from a holding potential of −10 mV, using a two-electrode voltage-clamp (GeneClamp 500B amplifier, Molecular Devices). They were filtered at 500 Hz and sampled at 4 kHz with a Digidata 1440A acquisition system (Molecular Devices). Oocytes were continuously perfused with control solution (in mM): 90 KCl, 1 MgCl₂, 1.8 CaCl₂, 5 HEPES (pH 7.4 with KOH) plus 3 mM Na-azide or 0.5 mM tolbutamide was added, as indicated.

### Patch-Clamp Recordings

Currents were recorded from giant inside-out patches excised from HEK cells expressing WT or mutant K_{ATP} channels, at a holding potential of −60 mV. Data were recorded with an Axopatch 200B amplifier (Molecular Devices), filtered at 1 kHz, and digitized at 10 kHz with a Digidata 1322A A/D interface driven by pClamp9 software (Molecular Devices).

For MgATP dose-response curves, the extracellular (pipette) solution contained (in mM): 140 KCl, 1.2 MgCl₂, 2.6 CaCl₂, 10 HEPES, pH 7.4 with KOH. The intracellular solution contained (in mM): 107 KCl, 2 MgCl₂, 1 CaCl₂, 10 EGTA, 10 HEPES, pH 7.3 with KOH. For ATP dose-response curves, the extracellular (pipette) solution contained (in mM): 140 KCl, 1 EDTA, 10 HEPES, pH 7.4 with KOH. The intracellular solution contained (in mM): 140 KCl, 1 EDTA, 1 EGTA, 10 HEPES, pH 7.3 with KOH. ATP sensitivity was determined by expressing the current in the test solution as a fraction of the mean of that in the control solution before and after each ATP application (3). ATP dose-response curves were individually fitted with the Hill equation: I/I_{C}=1/(1+([ATP]/IC₅₀)^{h}, where [ATP] is the ATP concentration; I_{C} and I are the current in the absence and presence of ATP, respectively; IC₅₀ is the ATP concentration producing half-maximal block of the K_{ATP} current and h is the hill coefficient.

Data were analysed with ClampFit (pClamp10; Molecular Devices) and Prism9 (GraphPad). Results are reported as mean±SEM and statistical significance determined using Student's t-test.

Three-Dimensional Modelling Kir6.2 three-dimensional modelling was undertaken using the reported human K_{ATP} bound to ATP and ADP in quatrefoil form (Protein Data Bank accession no. 6C3O) (4). Human SUR1 and Kir6.2, which share 95% and 97% identity respectively at amino acid level with their canine homologues, were used because canine K_{ATP} crystal structures are not available. Molecular modelling was performed using The PyMOL Molecular Graphics System (version 2.4.0, Schrödinger, LL Pymol).

### Example 2

The presence of rs851344999 was investigated in dogs outside the UK. The diabetes-associated T allele was not present in 230 non-diabetic controls of 9.

The rs851344999 SNP was subsequently included on a proprietary SNP-chip (the “Wisdom Panel”) to establish alternate allele frequency across a range of dog breeds in the USA undergoing breed ascertainment. Amongst 203,229 dogs of various breeds, 267 homozygote and 4,307 heterozygote individuals were identified amongst a random USA-based population, with an overall alternate allele frequency of 0.012. The majority of dogs carrying the alternate allele were of purebred Labrador retriever ancestry, although cross-bred dogs with Labrador retriever ancestry also carried the allele. The allele frequency in dogs classified as Labrador retrievers was 0.238.

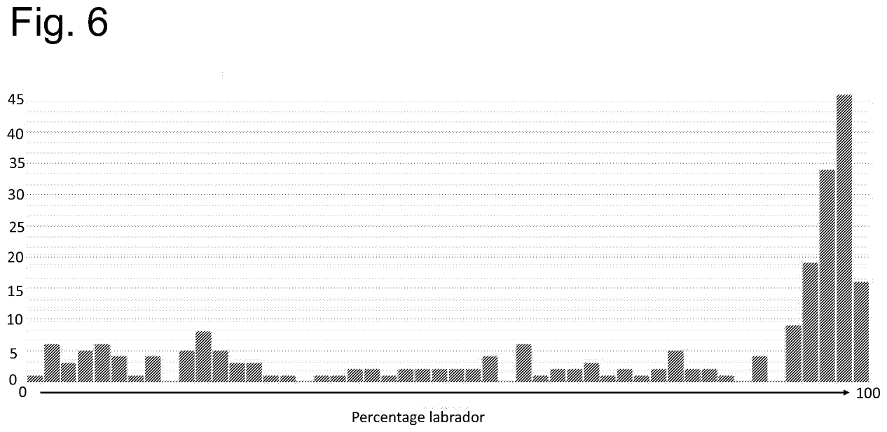

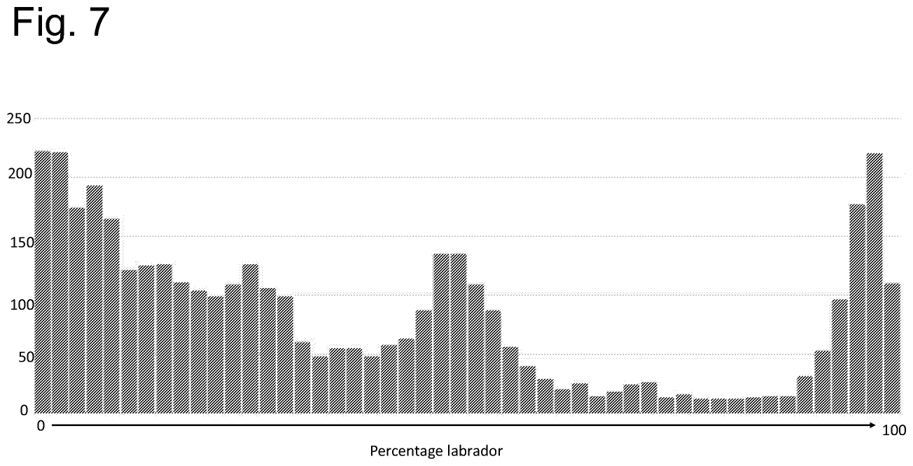

Of the 267 rs851344999 homozygotes, one dog had no detectable labrador retriever ancestry. Most dogs homozygous for the SNP have a large proportion of their genome which is consistent with Labrador breed ancestry (FIG. 6). As shown in FIG. 7, the dogs heterozygous for the rs851344999 SNP include Labradors retriever, as well as labrador retriever crosses (see peak in the middle of the histogram) and many dogs having a smaller proportion of Labrador retriever ancestry (towards the left of the histogram). Community science data for 434 of the homozygous and heterozygous dogs indicates that the median age at sampling was 2.8 years (range 0.2 to 16.5 years).

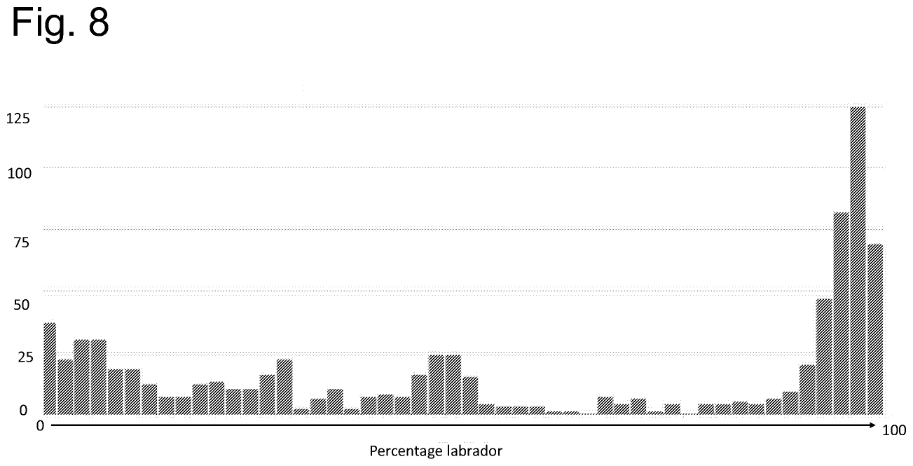

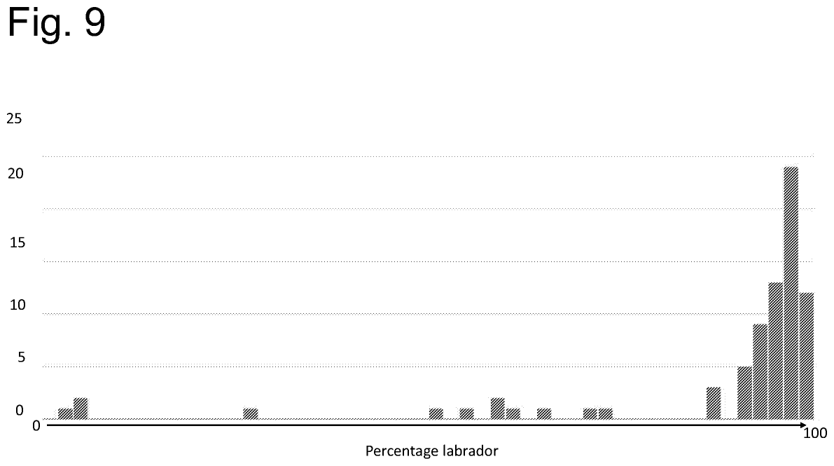

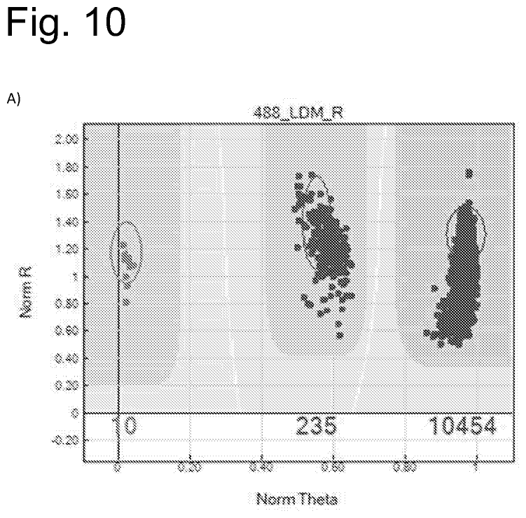

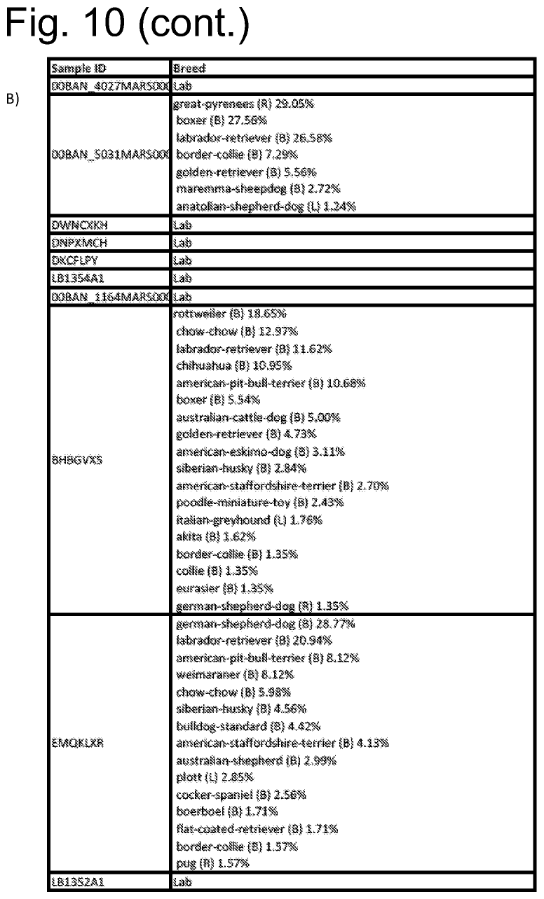

A subset of the dogs analysed using the Wisdom Panel is included in the Banfield Optimal Wellness Plans for puppies. It is thought that data from this subset may be more representative of the dog population in the US as a whole. Histograms of ancestry are provided in FIG. 8 (for heterozygous dogs) and FIG. 9 (for homozygous dogs). Community science data for 434 of the homozygous and heterozygous dogs indicates that the median age at sampling was 0.46 years (range 0.1 to 11.7 years). Full ancestry information for 10 representative homozygotes is shown in FIG. 10.

Information on the lifetime diabetes status of these dogs was not available.

1-8. (canceled)

9. A method of selecting a treatment for diabetes mellitus in a dog, comprising determining whether the genotype of the dog's KCNJ11 gene comprises a D274N allele, and selecting a treatment based on the determined genotype.

10. The method of claim 9, wherein an oral hypoglycaemic drug is selected as the treatment if the KCNJ11 genotype is homozygous or heterozygous for the D274N allele.

11. The method of claim 10, wherein the selected treatment further comprises injectable insulin.

12. The method of claim 9, wherein the selected treatment comprises injectable insulin if the KCNJ11 genotype is homozygous for a reference allele.

13. The method of claim 9, wherein the genotype of the KCNJ11 gene is determined by analysing a sample obtained from the dog.

14. The method of claim 13, wherein the sample is a blood sample or a tissue sample.

15. The method of claim 13, wherein the genotype of the KCNJ11 gene is determined by genome sequencing or by single nucleotide polymorphism (SNP) genotyping.

16. (canceled)

17. A method of preventing, delaying or treating diabetes mellitus in a dog whose KCNJ11 gene comprises one or more D274N alleles, comprising administering an oral hypoglycaemic drug to the dog.

18. (canceled)

19. The method of claim 17, wherein the KCNJ11 gene comprises two D274N alleles of the KCNJ11 gene.

20. The method of claim 9, wherein the dog is a Labrador retriever or has Labrador retriever ancestry, optionally wherein the dog is a flat coat retriever, a golden retriever, or a Labrador retriever-containing cross-breed.

21. The method of claim 9, wherein the diabetes mellitus is characterised by insulin dependence, pancreatic beta cell dysfunction and/or pancreatic beta cell loss.

22. The method of claim 10, wherein the oral hypoglycaemic drug is a sulphonylurea, tolbutamide, glibenclamide or glipizide.

23. (canceled)

24. A method of preventing or delaying diabetes mellitus in a dog whose KCNJ11 gene comprises one or more D274N alleles, comprising:
(a) providing the dog with an anti-hyperglycaemic diet; and/or
(b) neutering the dog, wherein the dog is female, optionally wherein neutering is performed before the dog's first oestrus cycle.

25. (canceled)

26. The method of claim 15, wherein the genome sequencing is Sanger sequencing, next generation sequencing (NGS), long read sequencing, single molecule real-time sequencing (SMRT), or DNA nanoball sequencing.

27. The method of claim 15, wherein the SNP genotyping comprises a 5′-nuclease allelic discrimination assay, restriction fragment length polymorphism analysis, allele-specific PCR, or a SNP array.

28. The method of claim 17, wherein the dog is a Labrador retriever or has Labrador retriever ancestry, optionally wherein the dog is a flat coat retriever, a golden retriever, or a Labrador retriever-containing cross-breed.

29. The method of claim 17, wherein the diabetes mellitus is characterised by insulin dependence, pancreatic beta cell dysfunction and/or pancreatic beta cell loss.

30. The method of claim 17, wherein the oral hypoglycaemic drug is a sulphonylurea, tolbutamide, glibenclamide or glipizide.
